SEQUENCE LISTING (40 sequences)

SEQ ID NO: 1 - RNA, 36 nt, synthetic construct
  misc_feature 1..36: note = Description of Artificial Sequence: Synthetic oligonucleotide
  modified_base 1: mod_base = OTHER; note = 2'-O-methyl modified nucleotide
  modified_base 1..2: mod_base = OTHER; note = Phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
  modified_base 26: mod_base = OTHER; note = 2'-O-methyl modified nucleotide
  modified_base 27: mod_base = OTHER; note = 2'-N-acetylgalactosamine conjugated nucleotide
  modified_base 28: mod_base = OTHER; note = 2'-N-acetylgalactosamine conjugated nucleotide
  modified_base 29: mod_base = OTHER; note = 2'-N-acetylgalactosamine conjugated nucleotide
  modified_base 30: mod_base = OTHER; note = 2'-N-acetylgalactosamine conjugated nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  source 1..36: mol_type = other RNA
atattttcccatctgtattagcagccgaaaggctgc

SEQ ID NO: 2 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 3 - RNA, 36 nt, synthetic construct; identical to SEQ ID NO: 1
  misc_feature 1..36: note = Description of Artificial Sequence: Synthetic oligonucleotide
  modified_base 1: mod_base = OTHER; note = 2'-O-methyl modified nucleotide
  modified_base 1..2: mod_base = OTHER; note = Phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-fluoro modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
  modified_base 26: mod_base = OTHER; note = 2'-O-methyl modified nucleotide
  modified_base 27: mod_base = OTHER; note = 2'-N-acetylgalactosamine conjugated nucleotide
  modified_base 28: mod_base = OTHER; note = 2'-N-acetylgalactosamine conjugated nucleotide
  modified_base 29: mod_base = OTHER; note = 2'-N-acetylgalactosamine conjugated nucleotide
  modified_base 30: mod_base = OTHER; note = 2'-N-acetylgalactosamine conjugated nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  source 1..36: mol_type = other RNA

SEQ ID NO: 4 - RNA, 36 nt, synthetic construct; identical to SEQ ID NO: 1
  misc_feature 1..36: note = Description of Artificial Sequence: Synthetic oligonucleotide
  modified_base 1: mod_base = OTHER; note = 2'-O-methyl modified nucleotide
  modified_base 1..2: mod_base = OTHER; note = Phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
  modified_base 26: mod_base = OTHER; note = 2'-O-methyl modified nucleotide
  modified_base 27: mod_base = OTHER; note = 2'-N-acetylgalactosamine conjugated nucleotide
  modified_base 28: mod_base = OTHER; note = 2'-N-acetylgalactosamine conjugated nucleotide
  modified_base 29: mod_base = OTHER; note = 2'-N-acetylgalactosamine conjugated nucleotide
  modified_base 30: mod_base = OTHER; note = 2'-N-acetylgalactosamine conjugated nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  source 1..36: mol_type = other RNA

SEQ ID NO: 5 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 6 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 7 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 8 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 9 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 10 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 11 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 12 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 13 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 14 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 15 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 16 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 17 - RNA, 22 nt, synthetic construct
  misc_feature 1..22: note = Description of Artificial Sequence: Synthetic oligonucleotide
  modified_base 1: mod_base = OTHER; note = 5'-vinylphosphonate 2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 18 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 19 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 20 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 21 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 22 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 23 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 24 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 25 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 26 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 27 - RNA, 36 nt, synthetic construct; identical to SEQ ID NO: 1
  misc_feature 1..36: note = Description of Artificial Sequence: Synthetic oligonucleotide
  modified_base 1: mod_base = OTHER; note = 2'-O-methyl modified nucleotide
  modified_base 1..2: mod_base = OTHER; note = Phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
  modified_base 26: mod_base = OTHER; note = 2'-O-methyl modified nucleotide
  modified_base 27: mod_base = OTHER; note = 2'-N-acetylgalactosamine conjugated nucleotide
  modified_base 28: mod_base = OTHER; note = 2'-N-acetylgalactosamine conjugated nucleotide
  modified_base 29: mod_base = OTHER; note = 2'-N-acetylgalactosamine conjugated nucleotide
  modified_base 30: mod_base = OTHER; note = 2'-N-acetylgalactosamine conjugated nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  source 1..36: mol_type = other RNA

SEQ ID NO: 28 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 29 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 30 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 31 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 32 - RNA, 36 nt, synthetic construct; identical to SEQ ID NO: 1
  misc_feature 1..36: note = Description of Artificial Sequence: Synthetic oligonucleotide
  modified_base 1: mod_base = OTHER; note = 2'-O-methyl modified nucleotide
  modified_base 1..2: mod_base = OTHER; note = Phosphorothioate linkage
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-fluoro modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
  modified_base 26: mod_base = OTHER; note = 2'-O-methyl modified nucleotide
  modified_base 27: mod_base = OTHER; note = 2'-N-acetylgalactosamine conjugated nucleotide
  modified_base 28: mod_base = OTHER; note = 2'-N-acetylgalactosamine conjugated nucleotide
  modified_base 29: mod_base = OTHER; note = 2'-N-acetylgalactosamine conjugated nucleotide
  modified_base 30: mod_base = OTHER; note = 2'-N-acetylgalactosamine conjugated nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  source 1..36: mol_type = other RNA

SEQ ID NO: 33 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 34 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-O-methyl modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 35 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 36 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-fluoro modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 37 - RNA, 22 nt, synthetic construct
  misc_feature 1..22: note = Description of Artificial Sequence: Synthetic oligonucleotide
  modified_base 1: mod_base = OTHER; note = 5'-phosphonate-4'-Oxy-2'-O-methyl modified nucleotide
  modified_base 1..2: mod_base = OTHER; note = Phosphorothioate linkage
  modified_base 2: mod_base = OTHER; note = 2'-fluoro modified nucleotide
  modified_base 2..3: mod_base = OTHER; note = Phosphorothioate linkage
  modified_base 3: mod_base = OTHER; note = 2'-fluoro modified nucleotide
  modified_base 3..4: mod_base = OTHER; note = Phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 20..21: mod_base = OTHER; note = Phosphorothioate linkage
  modified_base 21: mod_base = OTHER; note = 2'-O-methyl modified nucleotide
  modified_base 21..22: mod_base = OTHER; note = Phosphorothioate linkage
  modified_base 22: mod_base = OTHER; note = 2'-O-methyl modified nucleotide
  source 1..22: mol_type = other RNA
taatacagatgggaaaatatgg

SEQ ID NO: 38 - RNA, 36 nt, synthetic construct; identical to SEQ ID NO: 1
  misc_feature 1..36: note = Description of Artificial Sequence: Synthetic oligonucleotide
  source 1..36: mol_type = other RNA

SEQ ID NO: 39 - RNA, 22 nt, synthetic construct
  misc_feature 1..22: note = Description of Artificial Sequence: Synthetic oligonucleotide
  source 1..22: mol_type = other RNA
taatacagatgggaaaatatgg

SEQ ID NO: 40 - protein, 5 aa, synthetic construct
  REGION 1..5: note = Description of Artificial Sequence: Synthetic peptide
CREKA

(19) United States
(12) Patent Application Publication
(10) Pub. No.: US 2026/0125682 A1
(43) Pub. Date: May 7, 2026

(54) CHEMICAL MODIFICATIONS OF SMALL INTERFERING RNA WITH MINIMAL FLUORINE CONTENT

(71) Applicant: NOVO NORDISK A/S, Bagsvaerd (DK)

(72) Inventors: Weimin WANG, Cambridge, MA (US); Naim NAZEF, Lexington, MA (US)

(21) Appl. No.: 19/265,909

(22) Filed: Jul. 10, 2025

Related U.S. Application Data
(63) Continuation of application No. 17/766,153, now abandoned.
(60) Provisional application No. 62/909,278, filed on Oct. 2, 2019.

(51) Int. Cl. C12N 15/113

(52) U.S. Cl. CPC C12N 15/1137; C12N 2310/14; C12N 2310/321; C12N 2310/322; C12N 2310/351; C12N 2310/531

(57) ABSTRACT

The present invention provides oligonucleotides comprising 2′-O-methyl (2′-OMe) and 2′-deoxy-2′-fluoro (2′-F) modifications, compositions thereof, and methods of use for reducing the expression or activity of a gene.

### RELATED APPLICATIONS

The present application is a Continuation of U.S. application Ser. No. 17/766,153 filed Apr. 1, 2022, which is a § 371 National Stage of PCT International Application No. PCT/US2020/053999, filed Oct. 2, 2020, which claims priority under 35 U.S.C. § 119(c) from U.S. Provisional Patent Application No. 62/909,278, filed Oct. 2, 2019, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 10, 2025, is named 400930-002WO_176023_SL.txt and is 97,813 bytes in size.

### FIELD OF THE INVENTION

The present disclosure relates to oligonucleotides (e.g., RNA interference oligonucleotides) comprising 2′-O-methyl (2′-OMe) and 2′-deoxy-2′-fluoro (2′-F) modifications.

### BACKGROUND OF THE INVENTION

Oligonucleotides for reducing gene expression via RNA interference (RNAi) pathways have been developed. For example, RNAi oligonucleotides have been developed with each strand having sizes of 19-25 nucleotides with at least one 3′ overhang of 1 to 5 nucleotides (see, e.g., U.S. Pat. No. 8,372,968). Longer oligonucleotides have also been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as WO2010033225, which are incorporated herein by reference in their entirety). Such structures may include single-stranded extensions (on one or both sides of the molecule) as well as double-stranded extensions.

Chemical modification of such RNAi oligonucleotides is essential to fully harness the therapeutic potential of this class of molecules. Various chemical modifications have been developed and applied to RNAi oligonucleotides to improve their pharmacokinetics and pharmacodynamics properties (Deleavey & Damha, CHEM. BIOL., 19:937-954, 2012), and to block innate immune activation (Judge et al., MOL. THER., 13:494-505, 2006). One of the most common chemical modifications is to the 2′-OH of the furanose sugar of the ribonucleotides because of its involvement in the nuclease degradation. Fully chemically modified siRNAs with a combination of 2′-O-methyl (2′-OMe) and 2′-deoxy-2′-fluoro (2′-F) throughout the entire duplex have been reported and have demonstrated excellent stability and RNAi activity (Morrissey et al., HEPATOLOGY, 41:1349-1356, 2005; Allerson et al., J. MED. CHEM., 48:901-904, 2005; Hassler et al., NUCLEIC ACID RES., 46:2185-2196, 2018). More recently, N-acetylgalactosamine (GalNAc) conjugated chemically modified siRNAs have shown effective asialoglycoprotein receptor (ASGPr)-mediated delivery to liver hepatocytes in vivo (Nair et al., J. AM. CHEM. SOC., 136:16958-16961, 2014). Several GalNAc conjugated RNAi platforms including the GalNAc dicer-substrate conjugate (GalXC) platform, have advanced into clinical development for treating a wide range of human diseases.

One major concern with using chemically modified nucleoside analogues in the development of oligonucleotide-based therapeutics, including RNAi GalNAc conjugates, is the potential toxicity associated with the modifications. The therapeutic oligonucleotides could slowly degrade in patients, releasing nucleoside analogues that could be potentially phosphorylated and incorporated into cellular DNA or RNA. In the field of antivirus therapeutics, toxicity has emerged during the clinical development of many small molecule nucleotide inhibitors (Feng et al., ANTIMICROBIAL AGENTS AND CHEMOTHERAPY, 60:806-817, 2016). 2′-F modification of fully phosphorothioated antisense oligonucleotide has been reported to cause cellular protein reduction and double-stranded DNA breaks resulting in acute hepatotoxicity in vivo (Shen et al., Nucleic Acid Res., 43:4569-4578, 2015; Shen et al., NUCLEIC ACID RES., 46:2204-2217, 2018). No evidence has been observed so far for such liability of 2′-F modification in the context of RNAi oligonucleotides (Janas et al., NUCLEIC ACID THER., 26:363-371, 2016; Janas et al., NUCLEIC ACID THER., 27:11-22, 2016). Moreover, 2′-F siRNA have been well tolerated in clinical trials. Nonetheless, it is still desirable to minimize the use of unnatural nucleoside analogues such as 2′-F modified nucleosides in therapeutic RNA oligonucleotides.

Unlike 2′-deoxy-2′-fluoro RNA, 2′-O-Methyl RNA is a naturally occurring modification of RNA found in tRNA and other small RNAs that arise as a post-transcriptional modification. It is also known that the bulkier 2′-O-Methyl modification confers better metabolic stability as compared to the less bulky 2′-F modification. Therefore, 2′-OMe is preferable to 2′-F in terms of stability and tolerability. However, the bulkier 2′-OMe has been shown to interfere with RNA protein binding and inhibit RNAi activity if not positioned properly in the sequence of siRNA (Chiu et al., RNA, 9:1034-1048, 2003; Prakash et al., J. MED. CHEM., 48:4247-4253, 2005; Zheng et al., FASEB J., 27:4017-4026, 2013).

In order to further reduce the 2′-F content and increase the 2′-OMe content concomitantly so that the stability and tolerability can be improved without compromising RNAi activity, fine-tuning of the positions of the 2′-OMe and 2′-F (modification patterns) is necessary in DsiRNA conjugates that have already shown good potency and duration. A recent report has attempted to optimize modification patterns of a 21/23mer siRNA GalNAc conjugate platform (Foster et al., Mol. Ther. 26:708-717, 2018). That report, however, did not identify patterns of 2′-OMe and 2′-F that confer an oligonucleotide with high potency and duration as disclosed herein, including positions having poor tolerability to 2′-OMe substitution. Nor did that report identify advanced designs with minimal 2′-F content specifically for triloop and tetraloop GalXC platforms as disclosed herein.

### SUMMARY OF THE INVENTION

The present disclosure is based on the development of strategies for modifying an oligonucleotide (e.g., RNA interference oligonucleotide) with 2′-deoxy-2′-fluoro (2′-F) and 2′-O-Methyl (2′-OMe) modifications to increase its potency and duration.

Accordingly, aspects of the present disclosure provide an oligonucleotide comprising a sense strand comprising 17-36 nucleotides, wherein the sense strand has a first region (R1) and a second region (R2), wherein the second region of the sense strand comprises a first subregion (S1), a second subregion (S2) and a tetraloop (L) or triloop (triL) that joins the first and second regions, wherein the first and second regions form a second duplex (D2); an antisense strand comprising 20-22 nucleotides, wherein the antisense strand includes at least 1 single-stranded nucleotide at its 3′-terminus, wherein the sugar moiety of the nucleotides at position 5 of the antisense strand is modified with a 2′-F and the sugar moiety of each of the remaining nucleotides of the antisense strand is modified with a modification selected from the group consisting of 2′-O-propargyl, 2′-O-propylamin, 2′-amino, 2′-ethyl, 2′-aminoethyl (EA), 2′-fluoro (2′-F), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA), and wherein the sense strand and antisense strand are separate strands; and a first duplex (D1) formed by the first region of the sense strand and the antisense strand, wherein the first duplex has a length of 12-20 base pairs and has 7-10 nucleotides that are modified at the 2′-position of the sugar moiety with 2′-F.

The details of one or more embodiments of the disclosure are set forth in the description below. Other features or advantages of the present disclosure will be apparent from the detailed description of several embodiments and from the appended claims.

### BRIEF DESCRIPTION OF FIGURES

FIGS. 1A-1C shows data from a sense strand structure activity relationship (SAR). HAO1 target mRNA knockdown was measured at 48 hours after transfection of different concentrations of a nicked tetraloop GalNAc conjugate in a HAO1 stable cell line. Potency was determined as half maximal inhibitory concentration (IC₅₀). FIG. 1A is a graph showing potency of a sense strand in which positions 17 and 19 on the sense strand are modified with 2′-F. FIG. 1B is a graph showing potency of a sense strand in which position 19 of the sense strand is modified with 2′-F and position 17 of the sense strand is modified with 2′-OMe. FIG. 1C is a graph showing potency of a sense strand in which positions 17 and 19 on the sense strand are modified with 2′-OMe.

FIGS. 2A-2D shows data from an antisense strand structure activity relationship (SAR). HAO1 target mRNA knockdown was measured at 48 hours after transfection of different concentrations of a nicked tetraloop GalNAc conjugate in a HAO1 stable cell line. Potency was determined as half maximal inhibitory concentration (IC₅₀). FIG. 2A is a graph showing potency of an antisense strand in which positions 15, 17 and 19 on the sense strand are modified with 2′-F. FIG. 2B is a graph showing potency of an antisense strand in which positions 15 and 17 of the antisense strand are modified with 2′-F and position 19 of the antisense strand is modified with 2′-OMe. FIG. 2C is a graph showing potency of an antisense strand in which position 15 of the antisense strand is modified with 2′-F and positions 17 and 19 of the antisense strand are modified with 2′-OMe. FIG. 2D is a graph showing potency of an antisense strand in which positions 15, 17, and 19 of the antisense strand are modified with 2′-OMe.

FIGS. 3A-3H shows data from an antisense strand structure activity relationship (SAR). HAO1 target mRNA knockdown was measured at 48 hours after transfection of different concentrations of a nicked tetraloop GalNAc conjugate in a HAO1 stable cell line. Potency was determined as half maximal inhibitory concentration (IC₅₀). FIG. 3A is a graph showing potency of an antisense strand in which positions 1-3 and 5-10 of the antisense strand are modified with 2′-F and position 4 of the antisense strand is modified with 2′-OMe. FIG. 3B is a graph showing potency of an antisense strand in which positions 1-3, 5-8, and 10 of the antisense strand are modified with 2′-F and positions 4 and 9 of the antisense strand are modified with 2′-OMe. FIG. 3C is a graph showing potency of an antisense strand in which positions 1-3, 5-6, 8, and 10 of the antisense strand are modified with 2′-F and positions 4, 7, and 9 of the antisense strand are modified with 2′-OMe. FIG. 3D is a graph showing potency of an antisense strand in which positions 1-3, 6, 8, and 10 of the antisense strand are modified with 2′-F and positions 4, 5, 7, and 9 of the antisense strand are modified with 2′-OMe. FIG. 3E is a graph showing potency of an antisense strand in which positions 1-2, 6, 8, and 10 of the antisense strand are modified with 2′-F and positions 3, 4, 5, 7, and 9 of the antisense strand are modified with 2′-OMe. FIG. 3F is a graph showing potency of an antisense strand in which positions 1-2, 8, and 10 of the antisense strand are modified with 2′-F and positions 3-7 and 9 of the antisense strand are modified with 2′-OMe. FIG. 3G is a graph showing potency of an antisense strand in which positions 1-2 of the antisense strand are modified with 2′-F and positions 3-9 of the antisense strand are modified with 2′-OMe. FIG. 3H is a graph showing potency of an antisense strand in which positions 1-2 of the antisense strand are modified with 2′-F and positions 3-10 of the antisense strand are modified with 2′-OMe.

FIGS. 4A-4E shows data from an antisense strand structure activity relationship (SAR) in which modification with 2′-F at position 5 was maintained and positions 1-10 were probed with 2′-OMe. HAO1 target mRNA knockdown was measured at 48 hours after transfection of different concentrations of a nicked tetraloop GalNAc conjugate in a HAO1 stable cell line. Potency was determined as half maximal inhibitory concentration (IC₅₀). FIG. 4A is a graph showing potency of an antisense strand in which positions 1-3, 6, 8, 10, 14 and 15 of the antisense strand are modified with 2′-F and positions 4, 5, 7, 9, and 11-13 of the antisense strand are modified with 2′-OMe. FIG. 4B is a graph showing potency of an antisense strand in which positions 1-3, 6, 8, 10, and 14 of the antisense strand are modified with 2′-F and positions 4, 5, 7, 9, 11-13, and 15 of the antisense strand are modified with 2′-OMe. FIG. 4C is a graph showing potency of an antisense strand in which positions 1, 2, 6, 8, 10, 14 and 15 of the antisense strand are modified with 2′-F and positions 3-5, 7, 9, 11-13, and 15 of the antisense strand are modified with 2′-OMe. FIG. 4D is a graph showing potency of an antisense strand in which positions 2, 6, 8, 10, 14, and 15 of the antisense strand are modified with 2′-F and positions 1, 3-5, 7, 9, and 11-13 of the antisense strand are modified with 2′-OMe. FIG. 4E is a graph showing potency of an antisense strand in which positions 2 and 14 of the antisense strand are modified with 2′-F and positions 1, 3-13, and 15 of the antisense strand are modified with 2′-OMe.

FIGS. 5A-5H shows data from an antisense strand structure activity relationship (SAR) in which modification with 2′-F at positions 2 and 14 was maintained while addition of 2′-F was gradually made to the seed region at positions 3-6. HAO1 target mRNA knockdown was measured at 48 hours after transfection of different concentrations of a nicked tetraloop GalNAc conjugate in a HAO1 stable cell line. Potency was determined as half maximal inhibitory concentration (IC₅₀). FIG. 5A is a graph showing potency of an antisense strand in which positions 2 and 14 of the antisense strand are modified with 2′-F and positions 1 and 3-13 of the antisense strand are modified with 2′-OMe. FIG. 5B is a graph showing potency of an antisense strand in which positions 2, 3, and 14 of the antisense strand are modified with 2′-F and positions 1 and 4-13 of the antisense strand are modified with 2′-OMe. FIG. 5C is a graph showing potency of an antisense strand in which positions 2, 4, and 14 of the antisense strand are modified with 2′-F and positions 1, 3 and 5-13 of the antisense strand are modified with 2′-OMe. FIG. 5D is a graph showing potency of an antisense strand in which positions 2, 5, and 14 of the antisense strand are modified with 2′-F and positions 1, 3, 4, and 6-13 of the antisense strand are modified with 2′-OMe. FIG. 5E is a graph showing potency of an antisense strand in which positions 2, 6, and 14 of the antisense strand are modified with 2′-F and positions 1, 3-5, and 7-13 of the antisense strand are modified with 2′-OMe. FIG. 5F is a graph showing potency of an antisense strand in which positions 2, 3, 5, and 14 of the antisense strand are modified with 2′-F and positions 1, 4, and 6-13 of the antisense strand are modified with 2′-OMe. FIG. 5G is a graph showing potency of an antisense strand in which positions 2, 5, 6, and 14 of the antisense strand are modified with 2′-F and positions 1, 3, 4, and 7-13 of the antisense strand are modified with 2′-OMe. FIG. 5H is a graph showing potency of an antisense strand in which positions 2, 3, 5, 6, and 14 of the antisense strand are modified with 2′-F and positions 1, 4, and 7-13 of the antisense strand are modified with 2′-OMe.

FIGS. 6A-6F shows data from an antisense strand structure activity relationship (SAR) in which modification with 2′-F at positions 3 and 5 was maintained while addition of 2′-F was gradually made to positions 7-10. HAO1 target mRNA knockdown was measured at 48 hours after transfection of different concentrations of a nicked tetraloop GalNAc conjugate in a HAO1 stable cell line. Potency was determined as half maximal inhibitory concentration (IC₅₀). FIG. 6A is a graph showing potency of an antisense strand in which positions 1, 2, 3, 5 and 14 of the antisense strand are modified with 2′-F. FIG. 6B is a graph showing potency of an antisense strand in which positions 1, 2, 3, 5 and 14 of the antisense strand are modified with 2′-F and position 9 of the sense strand is modified with 2′-OMe. FIG. 6C is a graph showing potency of an antisense strand in which positions 1, 2, 3, 5, 7 and 14 of the antisense strand are modified with 2′-F and position 9 of the sense strand is modified with 2′-OMe. FIG. 6D is a graph showing potency of an antisense strand in which positions 1, 2, 3, 5, 8 and 14 of the antisense strand are modified with 2′-F and position 9 of the sense strand is modified with 2′-OMe. FIG. 6E is a graph showing potency of an antisense strand in which positions 1, 2, 3, 5, 9 and 14 of the antisense strand are modified with 2′-F and position 9 of the sense strand is modified with 2′-OMe. FIG. 6F is a graph showing potency of an antisense strand in which positions 1, 2, 3, 5, 10 and 14 of the antisense strand are modified with 2′-F and position 9 of the sense strand is modified with 2′-OMe.

FIGS. 7A-7H shows data from a structure activity relationship (SAR) of an antisense strand having minimal 2′-F modifications. HAO1 target mRNA knockdown was measured at 48 hours after transfection of different concentrations of a nicked tetraloop GalNAc conjugate in a HAO1 stable cell line. Potency was determined as half maximal inhibitory concentration (IC₅₀). FIG. 7A is a graph showing potency of an antisense strand in which positions 1, 2, 3, 5, 7, 9, 11, 13-15, 17 and 19 of the antisense strand are modified with 2′-F and positions 4, 6, 8, 10, 12, 16, and 18 of the antisense strand are modified with 2′-OMe, and a sense strand in which positions 3, 5, 7-13, 15, 17, and 19 of the sense strand are modified with 2′-F and positions 1, 2, 4, 6, 14, 16, and 18 of the sense strand are modified with 2′-OMe. FIG. 7B is a graph showing potency of an antisense strand in which positions 2, 5, and 14 of the antisense strand are modified with 2′-F and positions 1, 3, 4, and 6-13 of the antisense strand are modified with 2′-OMe, and a sense strand in which positions 8-11 of the sense strand are modified with 2′-F and positions 1-7 and 12-19 of the sense strand are modified with 2′-OMe. FIG. 7C is a graph showing potency of an antisense strand in which positions 1, 2, 5, and 14 of the antisense strand are modified with 2′-F and positions 3, 4, and 6-13 of the antisense strand are modified with 2′-OMe, and a sense strand in which positions 8-11 of the sense strand are modified with 2′-F and positions 1-7 and 12-19 of the sense strand are modified with 2′-OMe. FIG. 7D is a graph showing potency of an antisense strand in which positions 1-3, 5, 7, and 14 of the antisense strand are modified with 2′-F and positions 4, 6, and 8-13 of the antisense strand are modified with 2′-OMe, and a sense strand in which positions 8-11 of the sense strand are modified with 2′-F and positions 1-7 and 12-19 of the sense strand are modified with 2′-OMe. FIG. 7E is a graph showing potency of an antisense strand in which positions 1-3, 5, 10, and 14 of the antisense strand are modified with 2′-F and positions 4, 6-9, and 11-13 of the antisense strand are modified with 2′-OMe, and a sense strand in which positions 8-11 of the sense strand are modified with 2′-F and positions 1-7 and 12-19 of the sense strand are modified with 2′-OMe. FIG. 7F is a graph showing potency of an antisense strand in which positions 1-3, 5, 7, 9, and 14 of the antisense strand are modified with 2′-F and positions 4, 6, 8, and 10-13 of the antisense strand are modified with 2′-OMe, and a sense strand in which positions 8-11 of the sense strand are modified with 2′-F and positions 1-7 and 12-19 of the sense strand are modified with 2′-OMe. FIG. 7G is a graph showing potency of an antisense strand in which positions 1-3, 5, 7, 10, and 14 of the antisense strand are modified with 2′-F and positions 4, 6, 8, 9, and 11-13 of the antisense strand are modified with 2′-OMe, and a sense strand in which positions 8-11 of the sense strand are modified with 2′-F and positions 1-7 and 12-19 of the sense strand are modified with 2′-OMe. FIG. 7H is a graph showing potency of an antisense strand in which positions 2, 3, 5, 7, 10, and 14 of the antisense strand are modified with 2′-F and positions 4, 6, 8, 9, and 11-13 of the antisense strand are modified with 2′-OMe, and a sense strand in which positions 8-11 of the sense strand are modified with 2′-F and positions 1-7 and 12-19 of the sense strand are modified with 2′-OMe. FIG. 7I is a graph showing HAO1 mRNA expression in mice injected with an oligonucleotide depicted in FIGS. 7A-7H.

FIG. 8 is a graph showing HAO1 mRNA expression in mice injected with an oligonucleotide depicted in FIG. 18. Mice were injected with PBS as a control.

FIGS. 9A-9B show in vitro and in vivo data for an oligonucleotide set having minimal 2′-F modifications. FIG. 9A is a graph showing APOC3 mRNA expression in cells transfected with an oligonucleotide depicted in FIGS. 19A-19B. FIG. 9B is a graph showing APOC3 mRNA expression in mice injected with an oligonucleotide depicted in FIGS. 19A-19B. Mice were injected with PBS as a control.

FIG. 10 shows in vivo data for GYS2 dsRNAs with 3 GalNAc conjugated nucleotides in the loop region, and a high 2′-F modification pattern or one of the low 2′-F modification patterns labeled Pattern 1 or Pattern 2. Antisense strands contained either 3 phosphorothioates (3PS) or 2 phosphorothioates (2PS) at the 5′-end.

FIG. 11 shows the design of a series of dsRNA that were constructed to test the effects of replacing 2′-F with 2′-OMe.

FIG. 12 shows the design of dsRNA that were constructed to test the effects of replacing 2′-F with 2′-OMe at positions 15, 17, and 19 on the antisense strand.

FIGS. 13A-13B show the design of dsRNA that were constructed to test the effects of replacing 2′-F with 2′-OMe at positions 1-10 on the antisense strand.

FIG. 14 shows the design of dsRNA that were constructed to test the effects of replacing 2′-F with 2′-OMe at positions 1, 3, 6, 8, 10, and 15 on the antisense strand.

FIGS. 15A-15B show the design of dsRNA that were constructed to test the effects of the addition of 2′-F at positions 3-6 on the antisense strand.

FIGS. 16A-16B show the design of dsRNA that were constructed to test the effects of replacing 2′-F with 2′-OMe at positions 7 to 10, and maintaining 2′-F at positions 3 and 5 on the antisense strand.

FIGS. 17A-17B show the design of dsRNA that were constructed to test the in vivo activity of HAO1 conjugates comprising minimal 2′-F and heavy 2′-OMe modification patterns.

FIG. 18 shows the design of dsRNA that were constructed to test the duration of in vivo activity of HAO1 conjugates comprising minimal 2′-F and heavy 2′-OMe modification patterns.

FIGS. 19A-19B show the design of dsRNA that were constructed to test the in vitro and in vivo activity of APOC3 conjugates comprising the same minimal 2′-F and heavy 2′-OMe modification patterns as in the HAO1 conjugates shown in FIGS. 17A-17B.

FIGS. 20A-20B show the design of dsRNA that were constructed to test the in vitro and in vivo activity of GYS2 conjugates with three GalNAc conjugated nucleotides in the loop region and comprising the same minimal 2′-F and heavy 2′-OMe modification patterns as in the HAO1 conjugates shown in FIGS. 17A-17B.

### DETAILED DESCRIPTION OF THE INVENTION

Aspects of the present disclosure provide an oligonucleotide (e.g., RNA interference oligonucleotide) comprising modification patterns (e.g., 2′-deoxy-2′-fluoro (2′-F) and 2′-O-Methyl (2′-OMe) modification patterns) that alter an activity of the oligonucleotide compared to its unmodified counterpart. Accordingly, modification patterns provided herein may be useful for increasing binding of an oligonucleotide to its target (also known as oligonucleotide potency) and/or reducing binding of an oligonucleotide to a non-target (also known as off-target effects). In some embodiments, modification patterns provided herein may be useful for increasing resistance of an oligonucleotide to degradation and/or increasing duration of an oligonucleotide in a cell.

### (1) Definitions

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Administering: As used herein, the terms “administering” or “administration” means to provide a substance (e.g., an oligonucleotide) to a subject in a manner that is pharmacologically useful (e.g., to treat a condition in the subject). The oligonucleotides can also be administered by transfection or infection using methods known in the art, including but not limited to the methods described in McCaffrey et al. (2002), NATURE, 418 (6893), 38-9 (hydrodynamic transfection) or Xia et al. (2002), NATURE BIOTECHNOL., 20 (10), pp. 1006-10 (viral-mediated delivery);

Complementary: As used herein, the term “complementary” refers to a structural relationship between nucleotides (e.g., two nucleotide on opposing nucleic acids or on opposing regions of a single nucleic acid strand) that permits the nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have nucleotide sequences that are complementary to each other to form regions of complementarity, as described herein.

Deoxyribonucleotide: As used herein, the term “deoxyribonucleotide” refers to a nucleotide having a hydrogen at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

Double-stranded oligonucleotide: As used herein, the term “double-stranded oligonucleotide” refers to an oligonucleotide that is substantially in a duplex form. In some embodiments, complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from a single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequences of nucleotides that are partially complementary, and thus, may have one or more mismatches, which may include internal mismatches or end mismatches.

Duplex: As used herein, the term “duplex,” in reference to nucleic acids (e.g., oligonucleotides), refers to a structure formed through complementary base-pairing of two antiparallel sequences of nucleotides.

Excipient: As used herein, the term “excipient” refers to a non-therapeutic agent that may be included in a composition, for example, to provide or contribute to a desired consistency or stabilizing effect.

Loop: As used herein, the term “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

Modified Internucleotide Linkage: As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

Modified Nucleotide: As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modifications in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc. In certain embodiments, a modified nucleotide comprises a 2′-O-methyl or a 2′-F substitution at the 2′ position of the ribose ring.

Nicked Tetraloop Structure: A “nicked tetraloop structure” is a structure of a RNAi oligonucleotide characterized by the presence of separate sense (passenger) and antisense (guide) strands, in which the sense strand has a region of complementarity to the antisense strand such that the two strands form a duplex, and in which at least one of the strands, generally the sense strand, extends from the duplex in which the extension contains a tetraloop and two self-complementary sequences forming a stem region adjacent to the tetraloop, in which the tetraloop is configured to stabilize the adjacent stem region formed by the self-complementary sequences of the at least one strand.

Oligonucleotide: As used herein, the term “oligonucleotide” refers to a short nucleic acid, e.g., of less than 100 nucleotides in length. An oligonucleotide can comprise ribonucleotides, deoxyribonucleotides, and/or modified nucleotides including, for example, modified ribonucleotides. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

Overhang: As used herein, the term “overhang” refers to terminal non-base-pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotide.

Phosphate Analog: As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, for example, International Patent Application PCT/US2017/049909, filed on Sep. 1, 2017, U.S. Provisional Application No. 62/383,207, filed on Sep. 2, 2016, and 62/393,401, filed on Sep. 12, 2016, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al. (2015), NUCLEIC ACIDS RES., 43 (6): 2993-3011, the contents of each of which relating to phosphate analogs are incorporated herein by reference).

Reduced expression: As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with a double-stranded oligonucleotide (e.g., one having an antisense strand that is complementary to target mRNA sequence) may result in a decrease in the amount of RNA transcript, protein and/or enzymatic activity (e.g., encoded by the target gene) compared to a cell that is not treated with the double-stranded oligonucleotide. Similarly, “reducing expression” as used herein refers to an act that results in reduced expression of a gene (e.g., a target gene).

Region of Complementarity: As used herein, the term “region of complementarity” refers to a sequence of nucleotides of a nucleic acid (e.g., a double-stranded oligonucleotide) that is sufficiently complementary to an antiparallel sequence of nucleotides (e.g., a target nucleotide sequence within an mRNA) to permit hybridization between the two sequences of nucleotides under appropriate hybridization conditions, e.g., in a phosphate buffer, in a cell, etc. A region of complementarity may be fully complementary to a nucleotide sequence (e.g., a target nucleotide sequence present within an mRNA or portion thereof). For example, a region of complementary that is fully complementary to a nucleotide sequence present in an mRNA has a contiguous sequence of nucleotides that is complementary, without any mismatches or gaps, to a corresponding sequence in the mRNA. Alternatively, a region of complementarity may be partially complementary to a nucleotide sequence (e.g., a nucleotide sequence present in an mRNA or portion thereof). For example, a region of complementary that is partially complementary to a nucleotide sequence present in an mRNA has a contiguous sequence of nucleotides that is complementary to a corresponding sequence in the mRNA but that contains one or more mismatches or gaps (e.g., 1, 2, 3, or more mismatches or gaps) compared with the corresponding sequence in the mRNA, provided that the region of complementarity remains capable of hybridizing with the mRNA under appropriate hybridization conditions.

Ribonucleotide: As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

RNAi Oligonucleotide: As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA.

Strand: As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

Subject: As used herein, the term “subject” means any mammal, including mice, rabbits, and humans. In one embodiment, the subject is a human or non-human primate. The terms “individual” or “patient” may be used interchangeably with “subject.”

Synthetic: As used herein, the term “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

Targeting ligand: As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

Tetraloop: As used herein, the term “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base-pairing, stacking interactions, hydrogen bonding, and contact interactions (Cheong et al., NATURE 1990 Aug. 16; 346 (6285): 680-2; Heus and Pardi, SCIENCE 1991 Jul. 12; 253 (5016): 191-4). In some embodiments, a tetraloop comprises or consists of 3 to 6 nucleotides and is typically 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used as described in Cornish-Bowden (1985) NUCL. ACIDS RES. 13:3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., PROC NATL ACAD SCI USA. 1990 November; 87 (21): 8467-71; Antao et al., NUCLEIC ACIDS RES. 1991 Nov. 11; 19 (21): 5901-5). Examples of DNA tetraloops include the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)). See, for example: Nakano et al., BIOCHEMISTRY, 41 (48), 14281-292, 2002. Shinji et al. NIPPON KAGAKKAI KOEN YOKOSHU VOL. 78th; NO. 2; PAGE. 731 (2000), which are incorporated by reference herein for their relevant disclosures. In some embodiments, the tetraloop is contained within a nicked tetraloop structure.

Treat: As used herein, the term “treat” refers to the act of providing care to a subject in need thereof, e.g., through the administration a therapeutic agent (e.g., an oligonucleotide) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### (II) Oligonucleotides

One aspect of the present disclosure provides an oligonucleotide having a modification pattern that confers the oligonucleotide with increased potency and/or duration. As used herein, a modification pattern refers to an arrangement of modified nucleotides at certain positions in an oligonucleotide to enhance its potency and/or duration (e.g., modifications with 2′-F or 2′-OMe at certain positions in an oligonucleotide). Modification patterns disclosed herein may be incorporated into an oligonucleotide having any sequence (e.g., an oligonucleotide that targets any sequence) to enhance its potency and/or duration.

An oligonucleotide provided herein, in some embodiments, comprises a sense strand (also referred to as a passenger strand) and an antisense strand (also referred to as a guide strand) that are separate strands. In some embodiments, the sense strand has a first region (R1) and a second region (R2) that comprises a first subregion (S1), a second subregion (S2), and a tetraloop (L) or triloop (triL) that joins the first and second regions. In some embodiments, the first and second regions form a second duplex (D2). A second duplex (D2) may have various lengths. In some embodiments, the second duplex (D2) has a length of 1-6 base pairs. In some embodiments, the second duplex (D2) has a length of 2-6, 3-6, 4-6, 5-6, 1-5, 2-5, 3-5, or 4-5 base pairs. In some embodiments, the second duplex (D2) has a length of 1, 2, 3, 4, 5, or 6 base pairs.

In some embodiments, a first duplex (D1) is formed by the first region of the sense strand and the antisense strand. A first duplex (D1) may have various lengths. In some embodiments, the first duplex (D1) has a length of 12-20 base pairs. In some embodiments, the first duplex (D1) has a length of 13-20, 14-20, 15-20, 16-20, 17-20, 18-20, or 19-20 base pairs. In some embodiments, the first duplex (D1) has a length of 12-19, 12-18, 12-17, 12-16, 12-15, 12-14, or 12-13 base pairs in length. In some embodiments, the first duplex (D1) has a length of 12, 13, 14, 15, 16, 17, 18, 19, or 20 base pairs.

A first duplex (D1) or a second duplex (D2) may comprise at least one bicyclic nucleotide or locked nucleic acid (LNA). Locked nucleic acids, or LNAs, are well known to a skilled artisan (Elman et al., 2005; Kurreck et al., 2002; Crinelli et al., 2002; Braasch and Corey, 2001; Bondensgaard et al., 2000; Wahlestedt et al., 2000). In some embodiments, the first duplex (D1) comprises at least 1 bicyclic nucleotide. In some embodiments, the second duplex (D2) comprises at least 1 bicyclic nucleotide.

In some embodiments, an oligonucleotide provided herein comprising a sense strand and an antisense strand has an asymmetric structure. In some embodiments, an oligonucleotide has an asymmetric structure, with a sense strand having a length of 36 nucleotides, and an antisense strand having a length of 22 nucleotides with 2 single-stranded nucleotides at its 3′-terminus (also referred to as a 2 nucleotide 3′-overhang). In some embodiments, an oligonucleotide has an asymmetric structure, with a sense strand having a length of 35 nucleotides, and an antisense strand having a length of 21 nucleotides with 2 single-stranded nucleotides at its 3′-terminus. In some embodiments, an oligonucleotide has an asymmetric structure, with a sense strand having a length of 37 nucleotides, and an antisense strand having a length of 23 nucleotides with 2 single-stranded nucleotides at its 3′-terminus (also referred to as a 2 nucleotide 3′-overhang).

An oligonucleotide having an asymmetric structure as provided herein may include any length of single-stranded nucleotides at its 3′-terminus. In some embodiments, an oligonucleotide has an asymmetric structure, with a sense strand having a length of 36 nucleotides, and an antisense strand having a length of 22 nucleotides with 2 single-stranded nucleotide at its 3′-terminus. In some embodiments, an oligonucleotide has an asymmetric structure, with a sense strand having a length of 36 nucleotides, and an antisense strand having a length of 23 nucleotides with 3 single-stranded nucleotides at its 3′-terminus. In some embodiments, an oligonucleotide includes at least 1, at least 2, at least 3, at least 4, at least 5, at least 6, at least 7, at least 8, or more single-stranded nucleotides at its 3′-terminus. In some embodiments, an oligonucleotide includes 2, 3, 4, 5, 6, 7, 8, or more single-stranded nucleotides at its 3′-terminus.

In some embodiments, there is one or more (e.g., 1, 2, 3, 4, 5) mismatches between a sense and antisense strand in an oligonucleotide provided herein. If there is more than one mismatch between a sense and antisense strand, they may be positioned consecutively (e.g., 2, 3 or more in a row), or interspersed throughout the region of complementarity. In some embodiments, the first duplex (D1) contains one or more mismatches. In some embodiment, the second duplex (D2) contains one or more mismatches.

### (i) Antisense Strands

In some embodiments, an antisense strand of an oligonucleotide may be referred to as a “guide strand.” For example, if an antisense strand can engage with RNA-induced silencing complex (RISC) and bind to an Argonaute protein, or engage with or bind to one or more similar factors, and direct silencing of a target gene, it may be referred to as a guide strand. In some embodiments a sense strand complementary with a guide strand may be referred to as a “passenger strand.”

An antisense strand disclosed herein may comprise 20-22 nucleotides in length. In some embodiments, the antisense strand comprises 20-21 nucleotides in length or 21-22 nucleotides in length. In some embodiments, the antisense strand comprises 20 nucleotides in length, 21 nucleotides in length, or 22 nucleotides in length. In some embodiments, the antisense strand is 20 nucleotides in length, 21 nucleotides in length, or 22 nucleotides in length.

An oligonucleotide having an asymmetric structure as provided herein may include an antisense strand having any length of single-stranded nucleotides at its 3′-terminus. In some embodiments, the antisense strand includes at least 2 single-stranded nucleotides at its 3′-terminus. In some embodiments, the antisense strand includes at least 0, 1, 2, 3, at least 4, at least 5, at least 6 or more single-stranded nucleotides at its 3′-terminus. In some embodiments, the antisense strand includes 2 single-stranded nucleotides at its 3′-terminus. In some embodiments, the antisense strand includes 3 single-stranded nucleotides at its 3′-terminus. In some embodiments, the antisense strand includes 4 single-stranded nucleotides at its 3′-terminus. In some embodiments, the antisense strand includes 5 single-stranded nucleotides at its 3′-terminus. In some embodiments, the antisense strand includes 6 single-stranded nucleotides at its 3′-terminus.

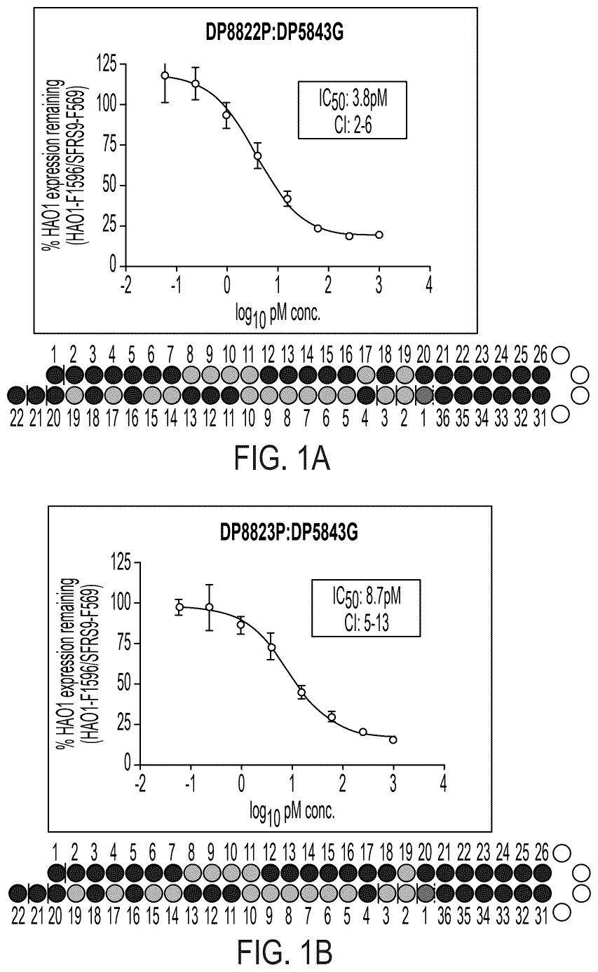

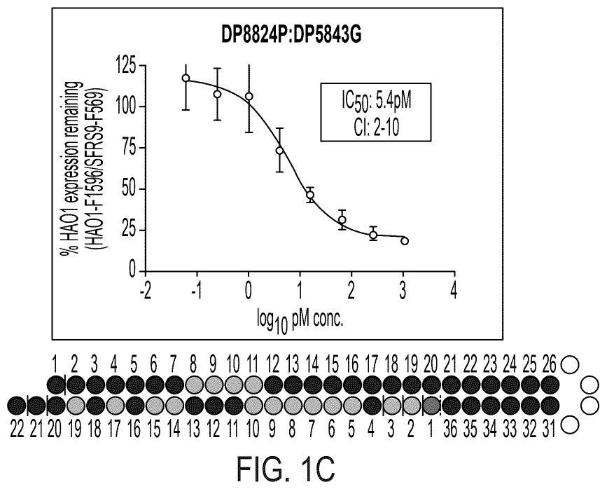

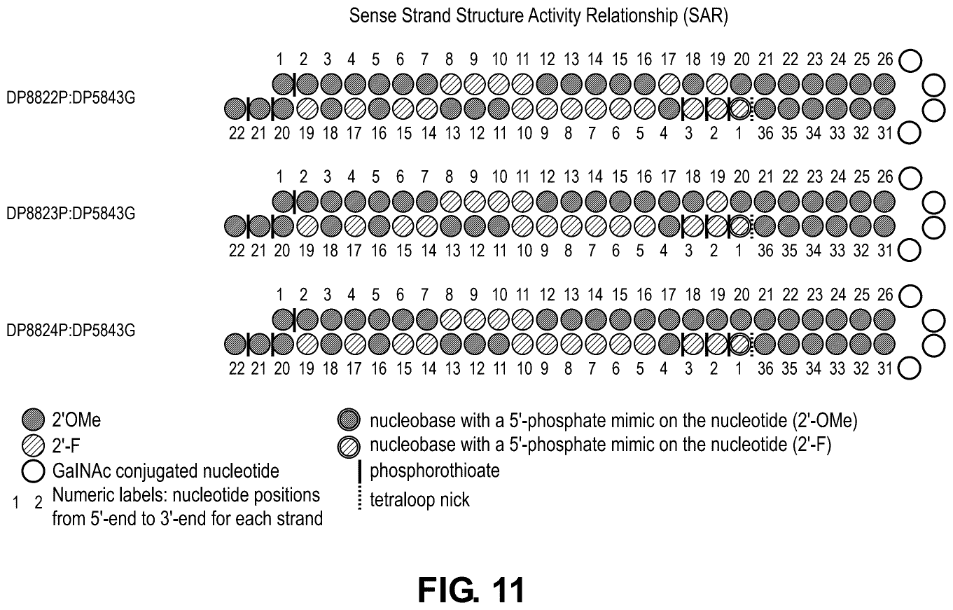

In some embodiments, an oligonucleotide disclosed herein comprises an antisense strand having nucleotides that are modified with 2′-F according to a modification pattern as set forth in any one of FIGS. 11-20B (as well as FIGS. 1-10). In some embodiments, an oligonucleotide disclosed herein comprises an antisense strand comprises nucleotides that are modified with 2′-F and 2′-OMe according to a modification pattern set forth in FIGS. 11-20B (as well as FIGS. 1-10). In some embodiments, an oligonucleotide provided herein comprises an antisense strand having the sugar moiety of the nucleotide at position 5 modified with 2′-F. In some embodiments, an oligonucleotide provided herein comprises an antisense strand having the sugar moiety of the nucleotide at position 5 modified with 2′-F and the sugar moiety of each of the remaining nucleotides of the antisense strand modified with a modification provided herein.

In some embodiments, an oligonucleotide provided herein comprises an antisense strand having the sugar moiety at positions 2 and 14 modified with 2′-F. In some embodiments, an oligonucleotide provided herein comprises an antisense strand having the sugar moiety at positions 2, 5, and 14 modified with 2′-F. In some embodiments, an oligonucleotide provided herein comprises an antisense strand having the sugar moiety at positions 1, 2, 5, and 14 modified with 2′-F. In some embodiments, an oligonucleotide provided herein comprises an antisense strand having the sugar moiety at positions 1, 2, 3, 5, 7, and 14 modified with 2′-F. In some embodiments, an oligonucleotide provided herein comprises an antisense strand having the sugar moiety at positions 1, 2, 3, 5, 10, and 14 modified with 2′-F.

In some embodiments, an oligonucleotide provided herein comprises an antisense strand having the sugar moiety of each of the nucleotides at positions 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

### (ii) Sense Strands

Oligonucleotides provided herein, in some embodiments, may comprise an antisense strand and a sense strand.

In some embodiments, a sense strand comprises 17-36 nucleotides in length. In some embodiments, a sense strand is 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, 31 nucleotides in length, 32 nucleotides in length, 33 nucleotides in length, 34 nucleotides in length, 35 nucleotides in length, or 36 nucleotides in length.

The sense strand, in some embodiments, has a first region (R1) and a second region (R2) that comprises a first subregion (S1) and a second subregion (S2) form a second duplex (D2). In some embodiments, a second duplex (D2) formed between a first subregion (S1) and a second subregion (S2) is at least 1 (e.g., at least 2, at least 3, at least 4, at least 5, or at least 6) base pairs in length. In some embodiments, a duplex formed between a first subregion (S1) and a second subregion (S2) is in the range of 1-6 base pairs in length (e.g., 1-5, 1-4, 1-3, 1-2, 2-6, 3-6, 4-6, or 5-6 base pairs in length).

In some embodiments, the second region (R2) comprises a tetraloop (L) or a triloop (triL) that joins the first and second regions. In some embodiments, the tetraloop or the triloop is at the 3′ terminus of the sense strand. In some embodiments, the tetraloop or the triloop is at the 5′ terminus of the antisense strand.

Any number of nucleotides in a triloop or a tetraloop may be conjugated to a targeting ligand. In some embodiments, a triloop comprises 1 nucleotide that is conjugated to a ligand. In some embodiments, a triloop comprises 2 nucleotides that are conjugated to a ligand. In some embodiments, a triloop comprises 3 nucleotides that are conjugated to a ligand. In some embodiments, a triloop comprises 1-3 nucleotides that are conjugated to a ligand. In some embodiments, a triloop comprises 1-2 nucleotides that are conjugated to a ligand or 2-3 nucleotides that are conjugated to a ligand.

In some embodiments, a tetraloop comprises 1 nucleotide that is conjugated to a ligand. In some embodiments, a tetraloop comprises 2 nucleotides that are conjugated to a ligand. In some embodiments, a tetraloop comprises 3 nucleotides that are conjugated to a ligand. In some embodiments, a tetraloop comprises 4 nucleotides that are conjugated to a ligand. In some embodiments, a tetraloop comprises 1˜4 nucleotides that are conjugated to a ligand. In some embodiments, a tetraloop comprises 1-3 nucleotides, 1-2 nucleotides, 2-4 nucleotides, or 3-4 nucleotides that are conjugated to a ligand.

In some embodiments, a tetraloop or a triloop may contain ribonucleotides, deoxyribonucleotides, modified nucleotides, and combinations thereof. Non-limiting examples of a RNA tetraloop include, but are not limited to, the UNCG family of tetraloops (e.g., UUCG), the GNRA family of tetraloops (e.g., GAAA), and the CUUG tetraloop. Non-limiting examples of, DNA tetraloops include, but are not limited to, the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)).

In some embodiments, an oligonucleotide disclosed herein comprises a sense strand having nucleotides that are modified with 2′-F according to a modification pattern as set forth in any one of FIGS. 11-20B (as well as FIGS. 1-10). In some embodiments, an oligonucleotide disclosed herein comprises a sense strand comprises nucleotides that are modified with 2′-F and 2′-OMe according to a modification pattern set forth in FIGS. 11-20B (as well as FIGS. 1-10).

In some embodiments, an oligonucleotide provided herein comprises a sense strand having the sugar moiety at positions 8-11 modified with 2′-F. In some embodiments, an oligonucleotide provided herein comprises a sense strand having the sugar moiety at positions 1-7 and 12-17 or 12-20 modified with 2′OMe. In some embodiments, an oligonucleotide provided herein comprises a sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-F.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-OMe.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

(iii) Oligonucleotide Modifications

Oligonucleotides may be modified in various ways to improve or control specificity, stability, delivery, bioavailability, resistance from nuclease degradation, immunogenicity, base-paring properties, RNA distribution and cellular uptake and other features relevant to therapeutic or research use. See, e.g., Bramsen et al., NUCLEIC ACIDS RES., 2009, 37, 2867-2881; Bramsen and Kjems (FRONTIERS IN GENETICS, 3 (2012): 1-22). Accordingly, some embodiments may include one or more suitable modifications. In some embodiments, a modified nucleotide has a modification in its base (or nucleobase), the sugar (e.g., ribose, deoxyribose), or the phosphate group.

The number of modifications on an oligonucleotide and the positions of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of its nucleotides to be modified. Accordingly, in certain embodiments of any of the oligonucleotides provided herein, all or substantially all the nucleotides of an oligonucleotide are modified. In certain embodiments, more than half of the nucleotides are modified. In certain embodiments, less than half of the nucleotides are modified. Typically, with naked delivery, every sugar is modified at the 2′-position. These modifications may be reversible or irreversible. In some embodiments, an oligonucleotide as disclosed herein has a number and type of modified nucleotides sufficient to cause the desired characteristic (e.g., protection from enzymatic degradation, capacity to target a desired cell after in vivo administration, and/or thermodynamic stability).

### (a) Sugar Modifications

In some embodiments, a modified sugar (also referred herein to a sugar analog) includes a modified deoxyribose or ribose moiety, e.g., in which one or more modifications occur at the 2′, 3′, 4′, and/or 5′ carbon position of the sugar. In some embodiments, a modified sugar may also include non-natural alternative carbon structures such as those present in locked nucleic acids (“LNA”) (see, e.g., Koshkin et al. (1998), TETRAHEDRON 54, 3607-3630), unlocked nucleic acids (“UNA”) (see, e.g., Snead et al. (2013), MOLECULAR THERAPY-NUCLEIC ACIDS, 2, c103), and bridged nucleic acids (“BNA”) (see, e.g., Imanishi and Obika (2002), The Royal Society of Chemistry, CHEM. COMMUN., 1653-1659). Koshkin et al., Snead et al., and Imanishi and Obika are incorporated by reference herein for their disclosures relating to sugar modifications.

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, the modification is 2′-fluoro, 2′-O-methyl, or 2′-O-methoxyethyl. In some embodiments a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the oligonucleotide described herein comprises at least one modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least one modified nucleotide (e.g., at least 1, at least 5, at least 10, at least 15, at least 20, at least 25, at least 30, at least 35, or more). In some embodiments, the antisense strand of the oligonucleotide comprises at least one modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′-fluoro or 2′-O-methyl, 2′-O-methoxyethyl, and 2′-deoxy-2′-fluoro-β-d-arabinonucleic acid). In some embodiments, the modified nucleotide comprises a 2′-modification (e.g., a 2′-fluoro or 2′-O-methyl)

The present disclosure provides oligonucleotides having different modification patterns. In some embodiments, the modified oligonucleotides comprise a sense strand sequence having a modification pattern as set forth in any one of FIGS. 11-20B (as well as FIGS. 1-10) and an antisense strand having a modification pattern as set forth in any one of FIGS. 11-20B (as well as FIGS. 1-10). In some embodiments, for these oligonucleotides, one or more of positions 8, 9, 10, or 11 of the sense strand is modified with a 2′-F group. In other embodiments, for these oligonucleotides, the sugar moiety at each of nucleotides at positions 1-7 and 12-20 in the sense strand is modified with a 2′-O-methyl.

In some embodiments, the present invention provide an oligonucleotide, which is, or comprises, a modified or unmodified sense strand selected from those listed in Table A. In some embodiments, the present invention provide an oligonucleotide, which is, or comprises, a modified or unmodified antisense strand selected from those listed in Table A. In some embodiments, the present invention provide a modified or unmodified double-stranded oligonucleotide selected from those listed in Table A. In some embodiments, the present invention provide a sense strand modification pattern selected from those listed in Table A. In some embodiments, the present invention provide an antisense strand modification pattern selected from those listed in Table A.

TABLE A
Sequence information for the oligonucleotides in FIGS. 11-18.

| DP number | | | SEQ | | SEQ |
|---|---|---|---|---|---|
| passenger: | | | ID | Corresponding | ID |
| Guide | Modification Pattern | sequence with Modifications | NO: | unmodified sequence | NO: |
| DP8822P: | {MS}MMMMMMFFFFMMMMM | [mAs][mU][mA][mU][mU][mU][mU][fC][fC][fC][fA][mU][mC][mU][mG][mU] | 1 | AUAUUUUCCCAUCUGUAUUA | 38 |
| DP5843G | FMFMMMMMMM[prg-peg- | [fA][mU][fU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}FMFMFFMMMFFF | [5VPfUs][fAs][fAs][mU][fA][fC][fA][fG][fA][fU][mG][mG][mG][fA][fA][mA] | 2 | UAAUACAGAUGGGAAAAUAUG | 39 |
| | FFFM{FS}{FS}{Px-FS} | [fA][mU][fA][mUs][mGs][mG] | | G | |
| DP8823P: | {MS}MMMMMMFFFFMMMMM | [mAs][mU][mA][mU][mU][mU][mU][fC][fC][fC][fA][mU][mC][mU][mG][mU] | 3 | AUAUUUUCCCAUCUGUAUUA | 38 |
| DP5843G | MMFMMMMMMM[prg-peg- | [mA][mU][fU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}FMFMFFMMMFFF | [5VPfUs][fAs][fAs][mU][fA][fC][fA][fG][fA][fU][mG][mG][mG][fA][fA][mA] | 2 | UAAUACAGAUGGGAAAAUAUG | 39 |
| | FFFM{FS}{FS}{Px-FS} | [fA][mU][fA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP5843G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}FMFMFFMMMFFF | [5VPfUs][fAs][fAs][mU][fA][fC][fA][fG][fA][fU][mG][mG][mG][fA][fA][mA] | 2 | UAAUACAGAUGGGAAAAUAUG | 39 |
| | FFFM{FS}{FS}{Px-FS} | [fA][mU][fA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9316G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}MMFMFFMMMFFF | [5VPfUs][fAs][fAs][mU][fA][fC][fA][fG][fA][fU][mG][mG][mG][fA][fA][mA] | 5 | UAAUACAGAUGGGAAAAUAUG | 39 |
| | FFFM{FS}{FS}{Px-FS} | [fA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9317G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMFFMMMFF | [5VPfUs][fAs][fAs][mU][fA][1C][fA][fG][fA][fU][mG][mG][mG][fA][fA][mA] | 6 | UAAUACAGAUGGGAAAAUAUG | 39 |
| | FFFFM{FS}{FS}{Px-FS} | [mA][mU][mA][mUs][mGs][mG] | | G | |
| DP8824P: | (MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9318G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMFF | [5VPfUs][fAs][fAs][mU][fA][fC][fA][fG][fA][fU][mG][mG][mG][fA][mA][mA] | 7 | UAAUACAGAUGGGAAAAUAUG | 39 |
| | FFFFM(FS}{FS}{Px-FS} | [mA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9320G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}MMMMFFMMMFM | [5VPfUs][fAs][fAs][mU][fA][fC][fA][fG][mA][fU][mG][mG][mG][fA][fA][mA] | 8 | UAAUACAGAUGGGAAAAUAUG | 39 |
| | FFFFM(FS}{FS}{Px-FS} | [mA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9321G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}MMMMFFMMMFM | [5VPfUs][fAs][fAs][mU][fA][fC][mA][fG][mA][fU][mG][mG][mG][fA][fA][mA] | 9 | UAAUACAGAUGGGAAAAUAUG | 39 |
| | FMFFM{FS}{FS}{Px-FS} | [mA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9322G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMFFMMMFM | [5VPfUs][fAs][fAs][mU][mA][fC][mA][fG][mA][fU][mG][mG][mG][fA][fA][mA] | 10 | UAAUACAGAUGGGAAAAUAUG | 39 |
| | FMFMM{FS}{FS}{Px-FS} | [mA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9323G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMFFMMMFM | [5VPfUs][fAs][mAs][mU][mA][fC][mA][fG][mA][fU][mG][mG][mG][fA][fA] | 11 | UAAUACAGAUGGGAAAAUAUG | 39 |
| | FMFMM{MS}{FS}{Px-FS} | [mA][mA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9324G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMFFMMMFM | [5VPfUs][fAs][mAs][mU][mA][mC][mA][fG][mA][fU][mG][mG][mG][fA][fA] | 12 | UAAUACAGAUGGGAAAAUAUG | 39 |
| | FMMMM{MS}{FS}{Px-FS} | [mA][mA][mU][mA][mUs][mGs][mG] | | G | |
| DP8824P: | (MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9325G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMFFMMMFM | [5VPfUs][fAs][mAs][mU][mA][mC][mA][mG][mA][fU][mG][mG][mG][fA][fA] | 13 | UAAUACAGAUGGGAAAAUAUG | 39 |
| | MMMMM{MS}{FS}{Px-FS} | [mA][mA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9326G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMFFMMMMM | [5VPfUs][fAs][mAs][mU][mA][mC][mA][mG][mA][mU][mG][mG][mG][fA][fA] | 14 | UAAUACAGAUGGGAAAAUAUG | 39 |
| | MMMMM{MS}{FS}{Px-FS} | [mA][mA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9319G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMFM | [5VPfUs][fAs][fAs][mU][mA][fC][mA][fG][mA][fU][mG][mG][mG][fA][mA] | 15 | UAAUACAGAUGGGAAAAUAUG | 39 |
| | FMFMM{FS}{FS}{Px-FS} | [mA][mA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9327G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMFFMMMFM | [5VPmUs][fAs][mAs][mU][mA][fC][mA][fG][mA][fU][mG][mG][mG][fA][fA] | 16 | UAAUACAGAUGGGAAAAUAUG | 39 |
| | FMFMM{MS}{FS}{Px-MS} | [mA][mA][mU][mA][mUs][mGs][mG] | | G | |
| DP8824P: | (MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP9328G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [5VPmUs][fAs][mAs][mU][mA][mC][mA][mG][mA][mU][mG][mG][mG][fA] | 17 | UAAUACAGAUGGGAAAAUAUG | 39 |
| | MMMMMM{MS}{FS}{Px-MS} | [mA][mA][mA][mU][mA][mUs][mGs][mG] | | G | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10016G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 18 | UAAUACAGAUGGGAAAAUAUG | 39 |
| | MMMMMM{MS}{FS}{Px-MS} | mUs][fAs][mAs][mU][mA][mC][mA][mG][mA][mU][mG][mG][mG][fA][mA] | | G | |
| | | [mA][mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10017G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 19 | UAAUACAGAUGGGAAAAUAUG | 39 |
| | MMMMMM{FS}{FS}{Px-MS} | mUs][fAs][fAs][mU][mA][mC][mA][mG][mA][mU][mG][mG][mG][fA][mA] | | G | |
| | | [mA][mA][mU][mA][mUs][mGs][mG] | | | |
| DP8824P: | (MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10024G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 20 | UAAUACAGAUGGGAAAAUAUG | 39 |
| | MMMMMF{MS}{FS}{Px-MS} | mUs][fAs][mAs][fU][mA][mC][mA][mG][mA][mU][mG][mG][mG][fA][mA] | | G | |
| | | [mA][mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10018G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}MMMMMFMMMM | [Phosphonate-4O- | 21 | UAAUACAGAUGGGAAAAUAUG | 39 |
| | MMMMFM{MS}{FS}{Px-MS} | mUs][fAs][mAs][mU][fA][mC][mA][mG][mA][mU][mG][mG][mG][fA][mA] | | G | |
| | | [mA][mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10025G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 22 | UAAUACAGAUGGGAAAAUAUG | 39 |
| | MMMFMM{MS}{FS}{Px-MS} | mUs][fAs][mAs][mU][mA][fC][mA][mG][mA][mU][mG][mG][mG][fA][mA] | | G | |
| | | [mA][mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10019G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 23 | UAAUACAGAUGGGAAAAUAUG | 39 |
| | MMMMFM{FS}{FS}{Px-MS} | mUs][fAs][fAs][mU][fA][mC][mA][mG][mA][mU][mG][mG][mG][fA][mA][mA] | | G | |
| | | [mA][mU][mA][mUs][mGs][mG] | | | |
| DP8824P: | (MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10026G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 24 | UAAUACAGAUGGGAAAAUAUG | 39 |
| | MMMFFM{MS}{FS}{Px-MS} | mUs][fAs][mAs][mU][fA][fC][mA][mG][mA][mU][mG][mG][mG][fA][mA][mA] | | G | |
| | | [mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10027G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}MMMMMFMMMM | [Phosphonate-4O- | 25 | UAAUACAGAUGGGAAAAUAUG | 39 |
| | MMMFFM{FS}{FS}{Px-MS} | mUs][fAs][fAs][mU][fA][fC][mA][mG][mA][mU][mG][mG][mG][fA][mA][mA] | | G | |
| | | [mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10633G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 26 | UAAUACAGAUGGGAAAAUAUG | 39 |
| | MMMMFM{FS}{FS}{Px-FS} | fUs][fAs][fAs][mU][fA][mC][mA][mG][mA][mU][mG][mG][mG][fA][mA][mA] | | G | |
| | | [mA][mU][mA][mUs][mGs][mG] | | | |
| DP10632P: | (MS}MMMMMMFMFFMMMMM | [mAs][mU][mA][mU][mU][mU][mU][fC][mC][fC][fA][mU][mC][mU][mG][mU] | 27 | AUAUUUUCCCAUCUGUAUUA | 38 |
| DP10633G | MMMMMMMMMMprg-peg- | [mA][mU][mU][mA][mG][mC][mA][mG][mC][mC][prgG-peg- | | GCAGCCGAAAGGCUGC | |
| | GalNAc][prg-peg-GalNAc][prg- | GalNAc][prgA-peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 26 | UAAUACAGAUGGGAAAAUAUG | 39 |
| | MMMMFM{FS}{FS}{Px-FS} | fUs][fAs][fAs][mU][fA][mC][mA][mG][mA][mU][mG][mG][mG][fA][mA][mA] | | G | |
| | | [mA][mU][mA][mUs][mGs][mG] | | | |
| DP10632P: | {MS}MMMMMMFMFFMMMMM | [mAs][mU][mA][mU][mU][mU][mU][fC][mC][fC][fA][mU][mC][mU][mG][mU] | 27 | AUAUUUUCCCAUCUGUAUUA | 38 |
| DP10634G | MMMMMMMMMM[prg-peg- | [mA][mU][mU][mA][mG][mC][mA][mG][mC][mC][prgG-peg- | | GCAGCCGAAAGGCUGC | |
| | GalNAc][prg-peg-GalNAc][prg- | GalNAc][prgA-peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}MMMMMFMMMM | [Phosphonate-4O- | 28 | UAAUACAGAUGGGAAAAUAUG | 39 |
| | MMFMFM{FS}{FS}{Px-FS} | fUs][fAs][fAs][mU][fA][mC][fA][mG][mA][mU][mG][mG][mG][fA][mA][mA] | | G | |
| | | [mA][mU][mA][mUs][mGs][mG] | | | |
| DP10632P: | {MS}MMMMMMFMFFMMMMM | [mAs][mU][mA][mU][mU][mU][mU][fC][mC][fC][fA][mU][mC][mU][mG][mU] | 27 | AUAUUUUCCCAUCUGUAUUA | 38 |
| DP10635G | MMMMMMMMMM[prg-peg- | [mA][mU][mU][mA][mG][mC][mA][mG][mC][mC][prgG-peg- | | GCAGCCGAAAGGCUGC | |
| | GalNAc][prg-peg-GalNAc][prg- | GalNAc][prgA-peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}MMMMMFMMMM | [Phosphonate-4O- | 29 | UAAUACAGAUGGGAAAAUAUG | 39 |
| | MFMMFM{FS}{FS}{Px-FS} | fUs][fAs][fAs][mU][fA][mC][mA][fG][mA][mU][mG][mG][mG][fA][mA][mA] | | G | |
| | | [mA][mU][mA][mUs][mGs][mG] | | | |
| DP10632P: | {MS}MMMMMMFMFFMMMMM | [mAs][mU][mA][mU][mU][mU][mU][fC][mC][fC][fA][mU][mC][mU][mG][mU] | 27 | AUAUUUUCCCAUCUGUAUUA | 38 |
| DP10636G | MMMMMMMMMM[prg-peg- | [mA][mU][mU][mA][mG][mC][mA][mG][mC][mC][prgG-peg- | | GCAGCCGAAAGGCUGC | |
| | GalNAc][prg-peg-GalNAc][prg- | GalNAc][prgA-peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMMF | [Phosphonate-4O- | 30 | UAAUACAGAUGGGAAAAUAUG | 39 |
| | MMMFM{FS}{FS}{Px-FS} | fUs][fAs][fAs][mU][fA][mC][mA][mG][fA][mU][mG][mG][mG][fA][mA][mA] | | G | |
| | | [mA][mU][mA][mUs][mGs][mG] | | | |
| DP10632P: | (MS}MMMMMMFMFFMMMMM | [mAs][mU][mA][mU][mU][mU][mU][fC][mC][fC][fA][mU][mC][mU][mG][mU] | 27 | AUAUUUUCCCAUCUGUAUUA | 38 |
| DP10637G | MMMMMMMMMMprg-peg- | [mA][mU][mU][mA][mG][mC][mA][mG][mC][mC][prgG-peg- | | GCAGCCGAAAGGCUGC | |
| | GalNAc][prg-peg-GalNAc][prg- | GalNAc][prgA-peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMFM | [Phosphonate-4O- | 31 | UAAUACAGAUGGGAAAAUAUG | 39 |
| | MMMFM{FS}{FS}{Px-FS} | fUs][fAs][fAs][mU][fA][mC][mA][mG][mA][fU][mG][mG][mG][fA][mA][mA] | | G | |
| | | [mA][mU][mA][mUs][mGs][mG] | | | |
| DP3692P: | (MS}MFMFMFFFFFFFMFMFM | [mAs][mU][fA][mU][fU][mU][fU][fC][fC][fC][fA][fU][fC][mU][fG][mU][fA] | 32 | AUAUUUUCCCAUCUGUAUUA | 38 |
| DP8180G | FMMMMMMMprg-peg- | [mU][fU][mA][mG][mC][mA][mG][mC][mC][prg G-peg-GalNAc][prgA-peg- | | GCAGCCGAAAGGCUGC | |
| | GalNAc][prg-peg-GalNAc][prg- | GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}FMFMFFFMFMFM | [Phosphonate-4O- | 33 | UAAUACAGAUGGGAAAAUAUG | 39 |
| | FMFM(FS}{FS}{Px-FS} | fUs][fAs][fAs][mU][fA][mC][fA][mG][fA][mU][fG][mG][fG][fA][fA][mA][fA] | | G | |
| | | [mU][fA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP11239G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 34 | UAAUACAGAUGGGAAAAUAUG | 39 |
| | MMMMFM{MS}{FS}{Px-FS} | fUs][fAs][mAs][mU][fA][mC][mA][mG][mA][mU][mG][mG][mG][fA][mA][mA] | | G | |
| | | [mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10634G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMM | [Phosphonate-4O- | 28 | UAAUACAGAUGGGAAAAUAUG | 39 |
| | MMFMFM{FS}{FS}{Px-FS} | fUs][fAs][fAs][mU][fA][mC][fA][mG][mA][mU][mG][mG][mG][fA][mA][mA] | | G | |
| | | [mA][mU][mA][mUs][mGs][mG] | | | |
| DP8824P: | (MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10637G | MMMMMMMMMMprg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMFM | [Phosphonate-4O- | 31 | UAAUACAGAUGGGAAAAUAUG | 39 |
| | MMMFM{FS}{FS}{Px-FS} | fUs][fAs][fAs][mU][fA][mC][mA][mG][mA][fU][mG][mG][mG][fA][mA][mA] | | G | |
| | | [mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP10638G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}MMMMMFMMMMF | [Phosphonate-4O- | 35 | UAAUACAGAUGGGAAAAUAUG | 39 |
| | MFMFM{FS}{FS}{Px-FS} | fUs][fAs][fAs][mU][fA][mC][fA][mG][fA][mU][mG][mG][mG][fA][mA][mA] | | G | |
| | | [mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP11240G | MMMMMMMMMM[prg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M{MS}{MS}MMMMMFMMMFM | [Phosphonate-4O- | 36 | UAAUACAGAUGGGAAAAUAUG | 39 |
| | MFMFM{FS}{FS}{Px-FS} | fUs][fAs][fAs][mU][fA][mC][fA][mG][mA][fU][mG][mG][mG][fA][mA][mA] | | G | |
| | | [mA][mU][mA][mUs][mGs][mG] | | | |
| DP8824P: | {MS}MMMMMMFFFFMMMMM | [mAs][mU][mA][mU][mU][mU][mU][fC][fC][fC][fA][mU][mC][mU][mG][mU] | 4 | AUAUUUUCCCAUCUGUAUUA | 38 |
| DP11244G | MMMMMMMMMMprg-peg- | [mA][mU][mU][mA][mG][mC][mA][mG][mC][mC][prgG-peg-GalNAc][prgA- | | GCAGCCGAAAGGCUGC | |
| | GalNAc][prg-peg-GalNAc][prg- | peg-GalNAc][prgA-peg-GalNAc][prgA-peg- | | | |
| | peg-GalNAc][prg-peg- | GalNAc][mG][mG][mC][mU][mG][mC] | | | |
| | GalNAc]MMMMMM | | | | |
| | M(MS}{MS}MMMMMFMMMFM | [Phosphonate-4O- | 37 | UAAUACAGAUGGGAAAAUAUG | 39 |
| | MFMFM{FS}{FS}{Px-MS} | mUs][fAs][fAs][mU][fA][mC][fA][mG][mA][fU][mG][mG][mG][fA][mA][mA] | | G | |
| | | [mA][mU][mA][mUs][mGs][mG] | | | |

In the modification patterns of Table A:
“M” refers to a 2′-OMe modified nucleotide;
“F” refers to a 2′-F modified nucleotide;
“S” refers to a nucleotide with a 3′-phosphorothioate linkage;
“{MS}” refers to a 2′-OMe modified nucleotide with a 3′-phosphorothioate linkage;
“{FS}” refers to a 2′-F modified nucleotide with a 3′-phosphorothioate linkage;
“[prg-peg-GalNAc]” refers to a nucleotide having a 2′-GalNAc conjugate:

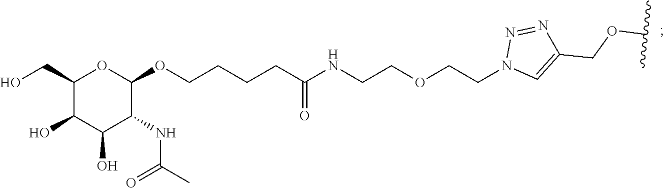 [SMILES: *OCc1cn(CCOCCNC(=O)CCCCO[C@@H]2O[C@H](CO)[C@H](O)[C@H](O)[C@H]2NC(C)=O)nn1]

“{Px-FS}” refers to a 2′-F modified nucleotide with a 3′-phosphorothioate linkage, and 5′ phosphonate or vinylphosphonate;
“{Px-MS}” refers to a 2′-OMe modified nucleotide with a 3′-phosphorothioate linkage, and 5′ phosphonate or vinylphosphonate.
   In the modified sequences of Table A:
“[mN]” refers to a 2′-OMe modified nucleotide;
“[fN]” refers to a 2′-F modified nucleotide;
“[Ns]” refers to a nucleotide with a 3′-phosphorothioate linkage;
“[mNs]” refers to a 2′-OMe modified nucleotide with a 3′-phosphorothioate linkage;
“[fNs]” refers to a 2′-F modified nucleotide with a 3′-phosphorothioate linkage;
“[prgG-peg-GalNAc]” refers to a G nucleotide having a 2′-GalNAc conjugate:

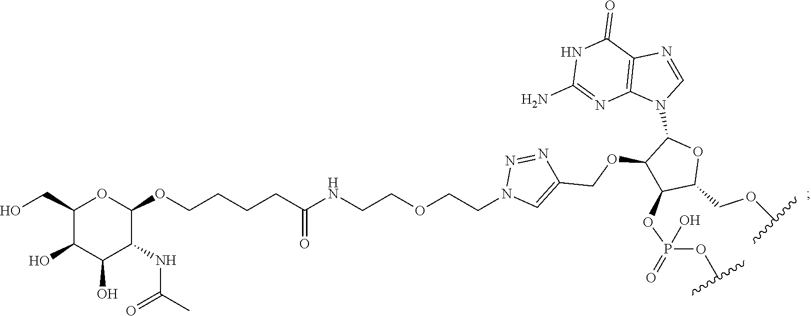 [SMILES: *OC[C@H]1O[C@@H](n2cnc3c(=O)[nH]c(N)nc32)[C@H](OCc2cn(CCOCCNC(=O)CCCCO[C@@H]3O[C@H](CO)[C@H](O)[C@H](O)[C@H]3NC(C)=O)nn2)[C@@H]1OP(=O)(O)O*]

“[prgA-peg-GalNAc]” refers to an A nucleotide having a 2′-GalNAc conjugate:

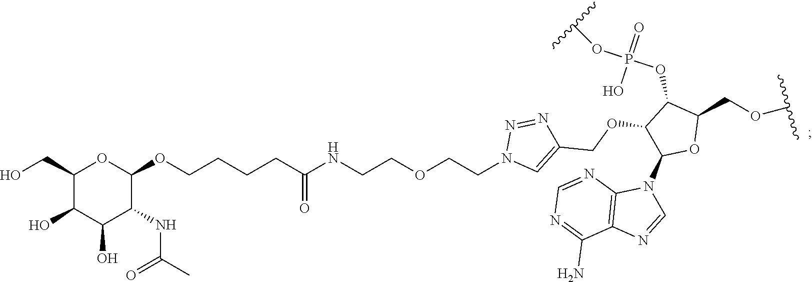 [SMILES: *OC[C@H]1O[C@@H](n2cnc3c(N)ncnc32)[C@H](OCc2cn(CCOCCNC(=O)CCCCO[C@@H]3O[C@H](CO)[C@H](O)[C@H](O)[C@H]3NC(C)=O)nn2)[C@@H]1OP(=O)(O)O*]

“[SVPfUs]” refers to a 5′-vinylphosphonate 2′-F uridine with a 3′-phosphorothioate linkage:

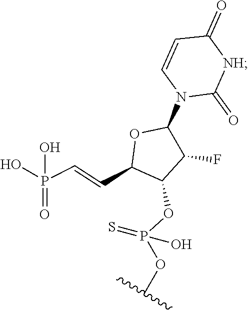 [SMILES: *OP(O)(=S)O[C@H]1[C@@H](F)[C@H](n2ccc(=O)[nH]c2=O)O[C@@H]1/C=C/P(=O)(O)O]

“[SVPmUs]” refers to a 5-vinylphosphonate 2′-OMe uridine with a 3′-phosphorothioate linkage:

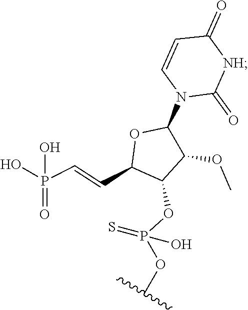 [SMILES: *OP(O)(=S)O[C@H]1[C@@H](OC)[C@H](n2ccc(=O)[nH]c2=O)O[C@@H]1/C=C/P(=O)(O)O]

“[Phosphonate-40-mUs]” refers to a 5′-phosphonate-4′-Oxy-2′-OMe uridine with a 3′-phosphorothioate linkage:

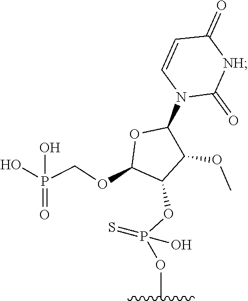 [SMILES: *OP(O)(=S)O[C@@H]1[C@@H](OCP(=O)(O)O)O[C@@H](n2ccc(=O)[nH]c2=O)[C@@H]1OC]

“[Phosphonate-40-fUs]” refers to a 5′-phosphonate-4′-Oxy-2′-F uridine with a 3′-phosphorothioate linkage:

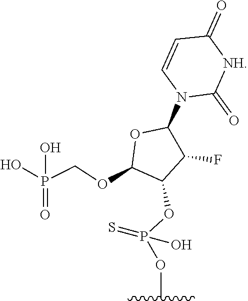 [SMILES: *OP(O)(=S)O[C@@H]1[C@@H](OCP(=O)(O)O)O[C@@H](n2ccc(=O)[nH]c2=O)[C@@H]1F]

In some embodiments, the antisense strand has 3 nucleotides that are modified at the 2′-position of the sugar moiety with a 2′-F. In some embodiments, the sugar moiety at positions 2, 5, and 14 and optionally up to 3 of the nucleotides at positions 1, 3, 7, and 10 of the antisense strand are modified with a 2′-F. In other embodiments, the sugar moiety at each of the positions at positions 2, 5, and 14 of the antisense strand is modified with the 2′-F. In other embodiments, the sugar moiety at each of the positions at positions 1, 2, 5, and 14 of the antisense strand is modified with the 2′-F. In still other embodiments, the sugar moiety at each of the positions at positions 1, 2, 3, 5, 7, and 14 of the antisense strand is modified with the 2′-F. In yet another embodiment, the sugar moiety at each of the positions at positions 1, 2, 3, 5, 10, and 14 of the antisense strand is modified with the 2′-F. In another embodiment, the sugar moiety at each of the positions at positions 2, 3, 5, 7, 10, and 14 of the antisense strand is modified with the 2′-F.

### (b) 5′ Terminal Phosphates

In some embodiments, 5′-terminal phosphate groups of oligonucleotides enhance the interaction with Argonaute 2. However, oligonucleotides comprising a 5′-phosphate group may be susceptible to degradation via phosphatases or other enzymes, which can limit their bioavailability in vivo. In some embodiments, oligonucleotides include analogs of 5′ phosphates that are resistant to such degradation. In some embodiments, a phosphate analog may be oxymethylphosphonate, vinylphosphonate, or malonylphosphonate. In certain embodiments, the 1′ end of an oligonucleotide strand is attached to chemical moiety that mimics the electrostatic and steric properties of a natural 5′-phosphate group (“phosphate mimic”).

In some embodiments, an oligonucleotide has a phosphate analog at a 4′-carbon position of the sugar (referred to as a “4′-phosphate analog”). Sec, for example, International Patent Application PCT/US2017/049909, filed on Sep. 1, 2017, U.S. Provisional Application Nos. 62/383,207, entitled 4′-Phosphate Analogs and Oligonucleotides Comprising the Same, filed on Sep. 2, 2016, and 62/393,401, filed on Sep. 12, 2016, entitled 4′-Phosphate Analogs and Oligonucleotides Comprising the Same, the contents of each of which relating to phosphate analogs are incorporated herein by reference. In some embodiments, an oligonucleotide provided herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or -O-CH₂-PO(OR)₂, in which R is independently selected from H, CH₃, an alkyl group, CH₂CH₂CN, CH₂OCOC(CH₃)₃, CH₂OCH₂CH₂Si(CH₃)₃, or a protecting group. In certain embodiments, the alkyl group is CH₂CH₃. More typically, R is independently selected from H, CH₃, or CH₂CH₃.

### (c). Modified Intranucleoside Linkages

In some embodiments, an oligonucleotide may comprise a modified internucleoside linkage. In some embodiments, phosphate modifications or substitutions may result in an oligonucleotide that comprises at least one (e.g., at least 1, at least 2, at least 3 or at least 5) modified internucleotide linkage. In some embodiments, any one of the oligonucleotides disclosed herein comprises 1 to 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9, or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, the oligonucleotide described herein has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand.

### (d) Base Modifications

In some embodiments, oligonucleotides provided herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom. See e.g., U.S. Published patent application No. 20080274462. In some embodiments, a modified nucleotide comprises a universal base. However, in certain embodiments, a modified nucleotide does not contain a nucleobase (abasic).

In some embodiments a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include inosine, 1-β-D-ribofuranosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside. NUCLEIC ACIDS RES. 1995 Nov. 11; 23 (21): 4363-70; Loakes et al., 3-Nitropyrrole and 5-nitroindole as universal bases in primers for DNA sequencing and PCR. NUCLEIC ACIDS RES. 1995 Jul. 11; 23 (13): 2361-6; Loakes and Brown, 5-Nitroindole as a universal base analogue, NUCLEIC ACIDS RES. 1994 Oct. 11; 22 (20): 4039-43. Each of the foregoing is incorporated by reference herein for their disclosures relating to base modifications).

### (e) Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See U.S. Published Application No. 2011/0294869 originally assigned to Traversa Therapeutics, Inc. (“Traversa”), PCT Publication No. WO 2015/188197 to Solstice Biologics, Ltd. (“Solstice”), Meade et al., NATURE BIOTECHNOLOGY, 2014, 32:1256-1263 (“Meade”), PCT Publication No. WO 2014/088920 to Merck Sharp & Dohme Corp, each of which are incorporated by reference for their disclosures of such modifications. This reversible modification of the internucleotide diphosphate linkages is designed to be cleaved intracellularly by the reducing environment of the cytosol (e.g. glutathione). Earlier examples include neutralizing phosphotriester modifications that were reported to be cleavable inside cells (Dellinger et al. J. AM. CHEM. SOC. 2003, 125:940-950).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of sugar, particularly when the modified nucleotide is the 3′-terminal nucleotide of the oligonucleotide. In some embodiments, the glutathione-sensitive moiety comprises a sulfonyl group. See, e.g., U.S. Prov. Appl. No. 62/378,635, entitled Compositions Comprising Reversibly Modified Oligonucleotides and Uses Thereof, which was filed on Aug. 23, 2016, and the contents of which are incorporated by reference herein for its relevant disclosures.

### (iv) Targeting Ligands

In some embodiments, it may be desirable to target the oligonucleotides of the disclosure to one or more cells or one or more organs. Such a strategy may help to avoid undesirable effects in other organs or may avoid undue loss of the oligonucleotide to cells, tissue or organs that would not benefit for the oligonucleotide. Accordingly, in some embodiments, oligonucleotides disclosed herein may be modified to facilitate targeting of a particular tissue, cell or organ, e.g., to facilitate delivery of the oligonucleotide to the liver. In certain embodiments, oligonucleotides disclosed herein may be modified to facilitate delivery of the oligonucleotide to the hepatocytes of the liver. In some embodiments, an oligonucleotide comprises a nucleotide that is conjugated to one or more targeting ligand.

A targeting ligand may comprise a carbohydrate, amino sugar, cholesterol, peptide, polypeptide, protein or part of a protein (e.g., an antibody or antibody fragment) or lipid. In some embodiments, a targeting ligand is an aptamer. For example, a targeting ligand may be an RGD peptide that is used to target tumor vasculature or glioma cells, CREKA peptide (SEQ ID NO: 40) to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., ligand are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand.

GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). Conjugation (either indirect or direct) of GalNAc moieties to oligonucleotides of the instant disclosure may be used to target these oligonucleotides to the ASGPR expressed on cells.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, an oligonucleotide of the instant disclosure is conjugated to a one or more bivalent GalNAc, trivalent GalNAc, or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of tetraloop are each conjugated to a separate GalNAc. In some embodiments, 1 to 3 nucleotides of triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, four GalNAc moieties can be conjugated to nucleotides in the tetraloop of the sense strand where each GalNAc moiety is conjugated to one nucleotide.

In some embodiments, an oligonucleotide herein comprises a monovalent GalNAc attached to a guanidine nucleotide, referred to as [ademG-GalNAc] or 2′-aminodiethoxymethanol-Guanidine-GalNAc, as depicted below:

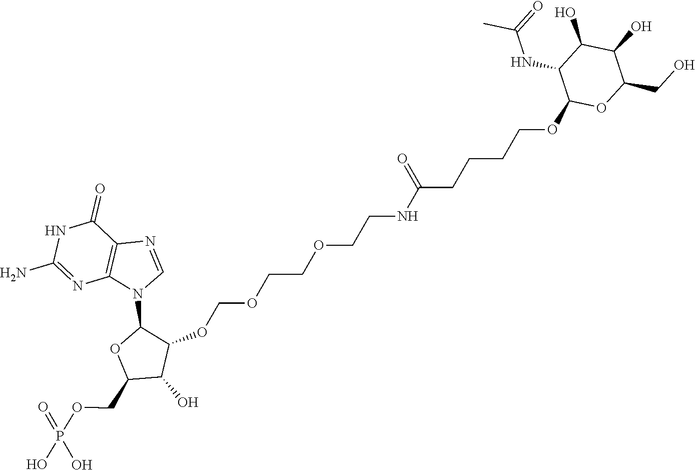 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below.

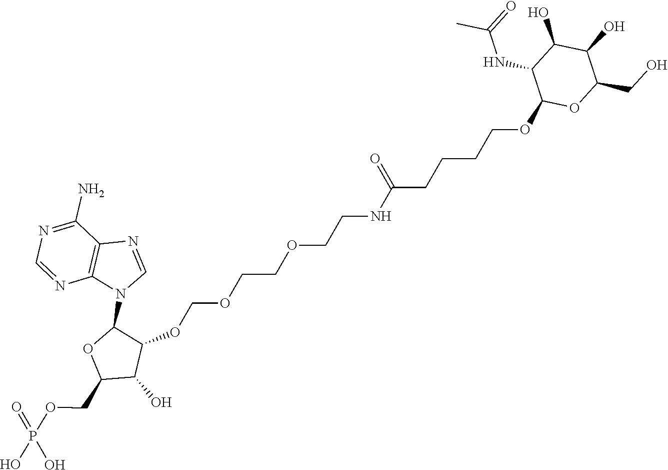

An example of such conjugation is shown below for a loop comprising from 5′ to 3′ the nucleotide sequence GAAA (L=linker, X=heteroatom) stem attachment points are shown. Such a loop may be present, for example, at positions 27-30 of the molecule shown in FIG. 1A. In the chemical formula,

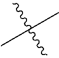

is used to describe an attachment point to the oligonucleotide strand.

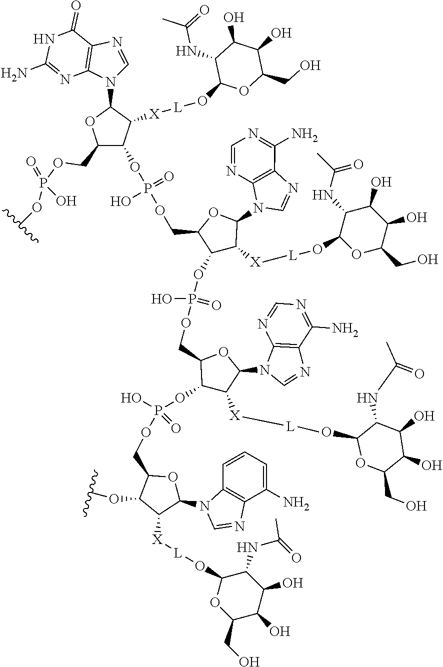

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International Patent Application Publication Number WO2016100401 A1, which published on Jun. 23, 2016, and the contents of which is incorporated herein by reference in its entirety. In some embodiments, the linker is a labile linker. However, in other embodiments, the linker is stable.

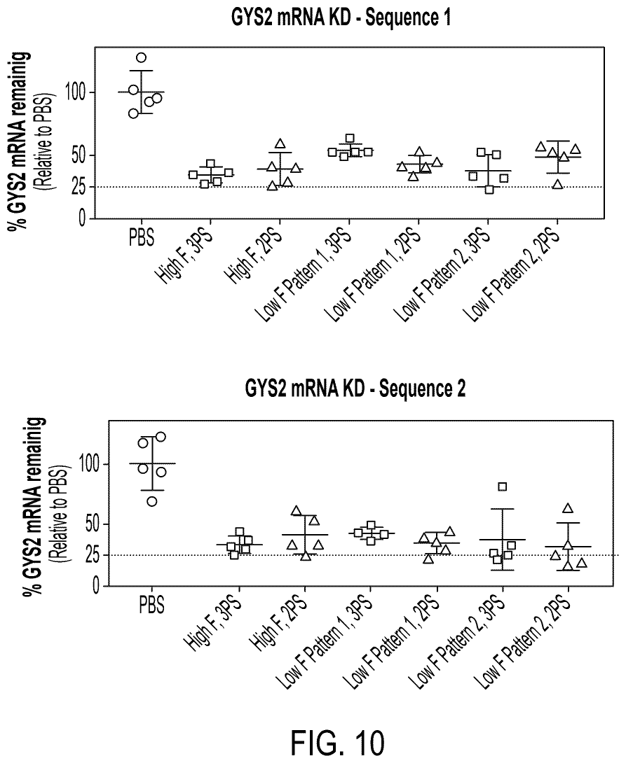

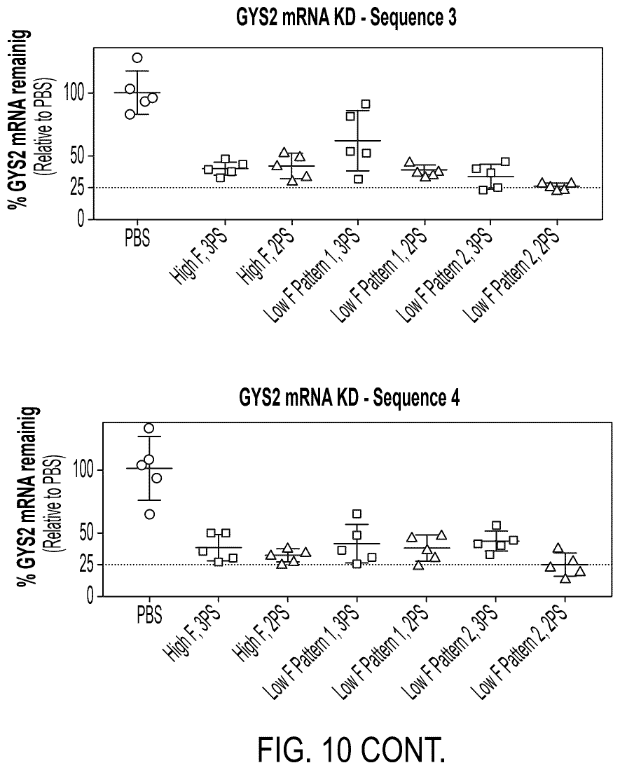

An example is shown below for a loop comprising from 5′ to 3′ the nucleotides GAAA, in which GalNac moieties are attached to nucleotides of the loop using an acetal linker. Such a loop may be present, for example, at positions 27-30 of the molecule shown in FIG. 10. In the chemical formula,

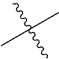

is an attachment point to the oligonucleotide strand.

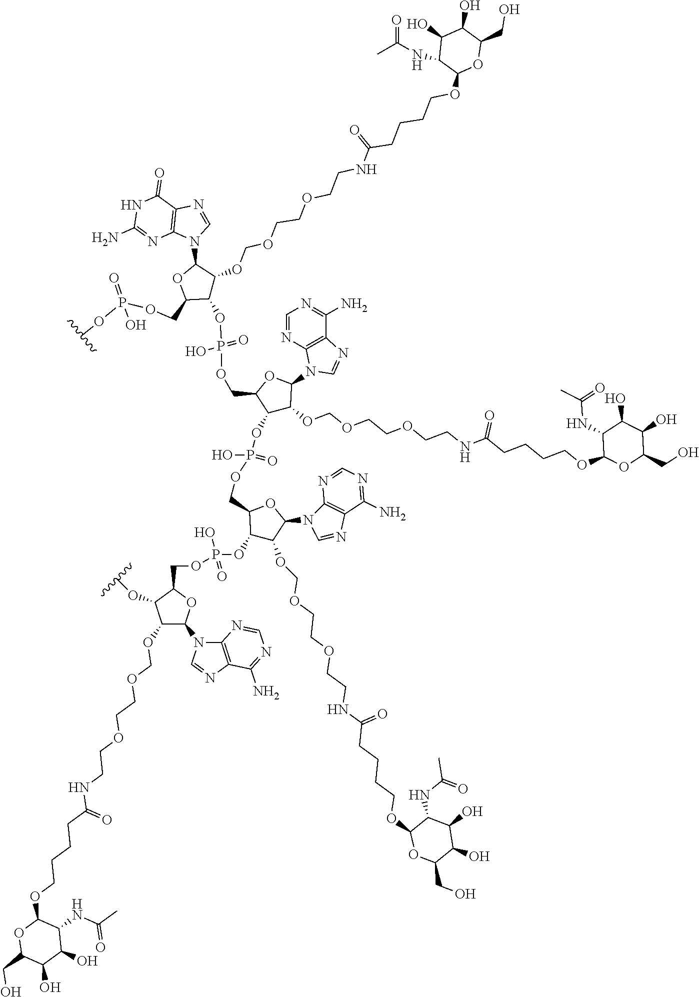

Any appropriate method or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International Patent Application Publication Number WO2016100401 A1, which published on Jun. 23, 2016, and the contents of which relating to such linkers are incorporated herein by reference. In some embodiments, the linker is a labile linker. However, in other embodiments, the linker is stable. A “labile linker” refers to a linker that can be cleaved, e.g., by acidic pH. A “fairly stable linker” refers to a linker that cannot be cleaved.

In some embodiments, a duplex extension (e.g., of up to 3, 4, 5, or 6 base pairs in length) is provided between a targeting ligand (e.g., a GalNAc moiety) and a double-stranded oligonucleotide. In some embodiments, the oligonucleotides of the present disclosure do not have a GalNAc conjugated.

### III. Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, an oligonucleotide is formulated in buffer solutions such as phosphate buffered saline solutions, liposomes, micellar structures, and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere, or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, formulations as disclosed herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol, or polyvinyl pyrolidone), or a or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.TM. (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent or more, although the percentage of the active ingredient(s) may be between about 1% 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though a number of embodiments are directed to liver-targeted delivery of any of the oligonucleotides disclosed herein, targeting of other tissues is also contemplated.

### IV. Methods of Use

### (a) Reducing RNA Expression in Cells

In some embodiments, methods are provided for delivering to a cell an effective amount any one of oligonucleotides disclosed herein for purposes of reducing expression of RNA in the cell. Methods provided herein are useful in any appropriate cell type. In some embodiments, a cell is any cell that expresses RNA (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue and skin). In some embodiments, the cell is a primary cell that has been obtained from a subject and that may have undergone a limited number of a passages, such that the cell substantially maintains is natural phenotypic properties. In some embodiments, a cell to which the oligonucleotide is delivered is ex vivo or in vitro (i.e., can be delivered to a cell in culture or to an organism in which the cell resides.

In some embodiments, oligonucleotides disclosed herein can be introduced using appropriate nucleic acid delivery methods including injection of a solution containing the oligonucleotides, bombardment by particles covered by the oligonucleotides, exposing the cell or organism to a solution containing the oligonucleotides, or electroporation of cell membranes in the presence of the oligonucleotides. Other appropriate methods for delivering oligonucleotides to cells may be used, such as lipid-mediated carrier transport, chemical-mediated transport, and cationic liposome transfection such as calcium phosphate, and others.

The consequences of inhibition can be confirmed by an appropriate assay to evaluate one or more properties of a cell or subject, or by biochemical techniques that evaluate molecules indicative of RNA expression (e.g., RNA, protein). In some embodiments, the extent to which an oligonucleotide provided herein reduces levels of expression of RNA is evaluated by comparing expression levels (e.g., mRNA or protein levels to an appropriate control (e.g., a level of RNA expression in a cell or population of cells to which an oligonucleotide has not been delivered or to which a negative control has been delivered). In some embodiments, an appropriate control level of RNAi expression may be a predetermined level or value, such that a control level need not be measured every time. The predetermined level or value can take a variety of forms. In some embodiments, a predetermined level or value can be single cut-off value, such as a median or mean.

In some embodiments, administration of an oligonucleotide as described herein results in a reduction in the level of RNA expression in a cell. In some embodiments, the reduction in levels of RNA expression may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of RNA. The appropriate control level may be a level of RNAi expression in a cell or population of cells that has not been contacted with an oligonucleotide as described herein. In some embodiments, the effect of delivery of an oligonucleotide to a cell according to a method disclosed herein is assessed after a finite period of time. For example, levels of RNA may be analyzed in a cell at least 8 hours, 12 hours, 18 hours, 24 hours; or at least one, two, three, four, five, six, seven, or fourteen days after introduction of the oligonucleotide into the cell.

In some embodiments, an oligonucleotide is delivered in the form of a transgene that is engineered to express in a cell the oligonucleotides (e.g., its sense and antisense strands). In some embodiments, an oligonucleotide is delivered using a transgene that is engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes can be injected directly to a subject.

### (b) Treatment Methods

Aspects of the disclosure relate to methods for reducing RNA expression in for attenuating the onset or progression of various diseases. In some embodiments, the disclosure provides methods for using RNAi oligonucleotides of the invention for treating subjects having or suspected of having liver conditions such as, for example, cholestatic liver disease, nonalcoholic fatty liver disease (NAFLD) and nonalcoholic steatohepatitis (NASH). In some embodiments, the disclosure provides RNAi oligonucleotides described herein for use in treating subjects having or suspected of having liver conditions such as, for example, cholestatic liver disease, NAFLD and NASH. In some embodiments, the disclosure provides RNAi for the preparation of a medicament for treatment of subjects having or suspected of having liver conditions such as, for example, cholestatic liver disease, NAFLD and nonalcoholic steatohepatitis NASH.

In a further aspect, the present invention relates to a method for treating a subject having a disease or at risk of developing a disease caused by the expression of a target gene. In this embodiment, the oligonucleotides can act as novel therapeutic agents for controlling one or more of cellular proliferative and/or differentiative disorders, disorders associated with bone metabolism, immune disorders, hematopoietic disorders, cardiovascular disorders, liver disorders, viral diseases, or metabolic disorders. The method comprises administering a pharmaceutical composition of the invention to the patient (e.g., human), such that expression of the target gene is silenced. Because of their high specificity, the oligonucleotides of the present invention specifically target mRNAs of target genes of diseased cells and tissues.

In the prevention of disease, the target gene may be one which is required for initiation or maintenance of the disease, or which has been identified as being associated with a higher risk of contracting the disease. In the treatment of disease, the oligonucleotide can be brought into contact with the cells or tissue exhibiting the disease. For example, oligonucleotide substantially identical to all or part of a mutated gene associated with cancer, or one expressed at high levels in tumor cells, e.g., aurora kinase, may be brought into contact with or introduced into a cancerous cell or tumor gene.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin. As used herein, the terms “cancer,” “hyperproliferative,” and “neoplastic” refer to cells having the capacity for autonomous growth, i.e., an abnormal state of condition characterized by rapidly proliferating cell growth. These terms are meant to include all types of cancerous growths or oncogenic processes, metastatic tissues or malignantly transformed cells, tissues, or organs, irrespective of histopathologic type or stage of invasiveness. Proliferative disorders also include hematopoietic neoplastic disorders, including diseases involving hyperplastic/neoplastic cells of hematopoietic origin, e.g., arising from myeloid, lymphoid or erythroid lineages, or precursor cells thereof.

The present invention can also be used to treat a variety of immune disorders, in particular those associated with overexpression of a gene or expression of a mutant gene. Examples of hematopoietic disorders or diseases include, without limitation, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus crythematosus, autoimmune thyroiditis, dermatitis (including atopic dermatitis and eczematous dermatitis), psoriasis, Sjogren's Syndrome, Crohn's disease, aphthous ulcer, iritis, conjunctivitis, kerato-conjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing, loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy.

In another embodiment, the invention relates to a method for treating viral diseases, including but not limited to human papilloma virus, hepatitis C, hepatitis B, herpes simplex virus (HSV), HIV-AIDS, poliovirus, and smallpox virus. Oligonucleotides of the invention are prepared as described herein to target expressed sequences of a virus, thus ameliorating viral activity and replication. The molecules can be used in the treatment and/or diagnosis of viral infected tissue, both animal and plant. Also, such molecules can be used in the treatment of virus-associated carcinoma, such as hepatocellular cancer.

The oligonucleotide of the present invention can also be used to inhibit the expression of the multi-drug resistance 1 gene (“MDR1”). “Multi-drug resistance” (MDR) broadly refers to a pattern of resistance to a variety of chemotherapeutic drugs with unrelated chemical structures and different mechanisms of action. Although the etiology of MDR is multifactorial, the overexpression of P-glycoprotein (Pgp), a membrane protein that mediates the transport of MDR drugs, remains the most common alteration underlying MDR in laboratory models (Childs and Ling, 1994). Moreover, expression of Pgp has been linked to the development of MDR in human cancer, particularly in the leukemias, lymphomas, multiple myeloma, neuroblastoma, and soft tissue sarcoma (Fan et al.). Recent studies showed that tumor cells expressing MDR-associated protein (MRP) (Cole et al., 1992), lung resistance protein (LRP) (Scheffer et al., 1995) and mutation of DNA topoisomerase II (Beck, 1989) also may render MDR.

In some embodiments, the target gene may be a target gene from any mammal, such as a human target. Any gene may be silenced according to the method described herein. Exemplary target genes include, but are not limited to, Factor VII, Eg5, PCSK9, TPX2, apoB, LDHA, SAA, TTR, HBV, HCV, RSV, PDGF beta gene, Erb-B gene, Src gene, CRK gene, GRB2 gene, RAS gene, MEKK gene, JNK gene, HMGB1 gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, p73 gene, p21(WAFl/CIPl) gene, p27(KIPl) gene, PPM1D gene, HAO1 gene, RAS gene, caveolin I gene, MIB I gene, MTAI gene, M68 gene, mutations in tumor suppressor genes, p53 tumor suppressor gene, LDHA, HMGB1, HAO1, and combinations thereof.

Methods described herein are typically involved administering to a subject in an effective amount of an oligonucleotide, that is, an amount capable of producing a desirable therapeutic result. A therapeutically acceptable amount may be an amount that is capable of treating a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions disclosed herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides disclosed herein are administered intravenously or subcutaneously.

As a non-limiting set of examples, the oligonucleotides of the instant disclosure would typically be administered quarterly (once every three months), bi-monthly (once every two months), monthly, or weekly. For example, the oligonucleotides may be administered every week or at intervals of two, or three weeks. The oligonucleotides may be administered daily.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### EXAMPLES

In order that the invention described herein may be more fully understood, the following examples are set forth. The examples described in this application are offered to illustrate the methods, compositions, and systems provided herein and are not to be construed in any way as limiting their scope.

### Example 1: Sense Strand Analyzed by Replacing 2′-F with 2′-OMe at Positions 17 and 19

A double stranded RNA (dsRNA) that targets HAO1 was selected for structure activity relationship (SAR) analysis. The dsRNA comprises a tetraloop, where each base is conjugated to a simple sugar, N-acetylgalactosamine (GalNAc). The sense and antisense strands of the dsRNA are modified with 2′-F at positions 8-11 and at positions 2 and 14, respectively. These modifications increased RNAi potency as compared to the dsRNA modified with 2′-OMe at the same positions. Accordingly, the just-noted 2′-F modifications were held constant during SAR described herein.

To test the effects of replacing 2′-F with 2′-OMe, a series of dsRNA were constructed as shown in FIG. 11. To analyze potency of the dsRNA, HAO1 mRNA knockdown was measured at 48 hours after transfection of different concentrations of dsRNA in a HAO1 stable cell line. Potency was then calculated as half maximal inhibitory concentration (IC₅₀). Similar potency was determined for each of the tested dsRNA as shown in FIGS. 1A-1C. Taken together, these results demonstrate that 2′-OMe modifications are well tolerated on the sense strand of the dsRNA.

### Example 2: Antisense Strand Analyzed by Replacing 2′-F with 2′-OMe at Positions 15, 17, and 19

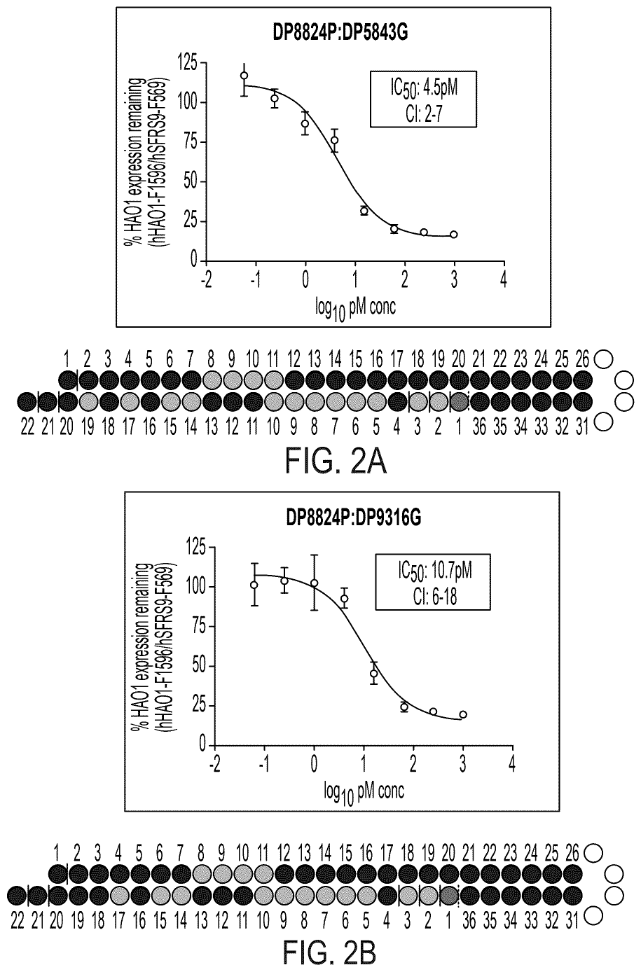

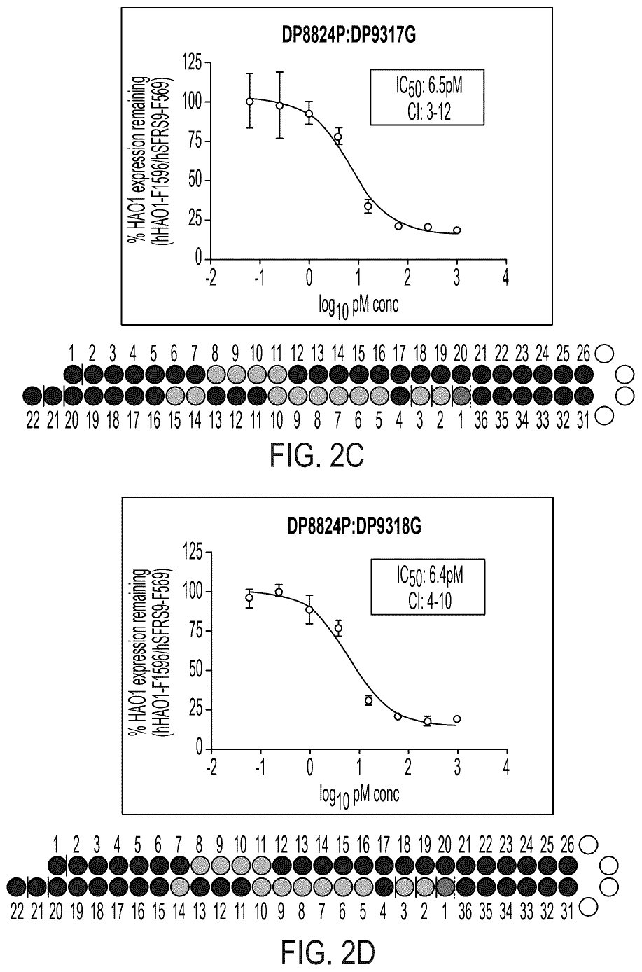

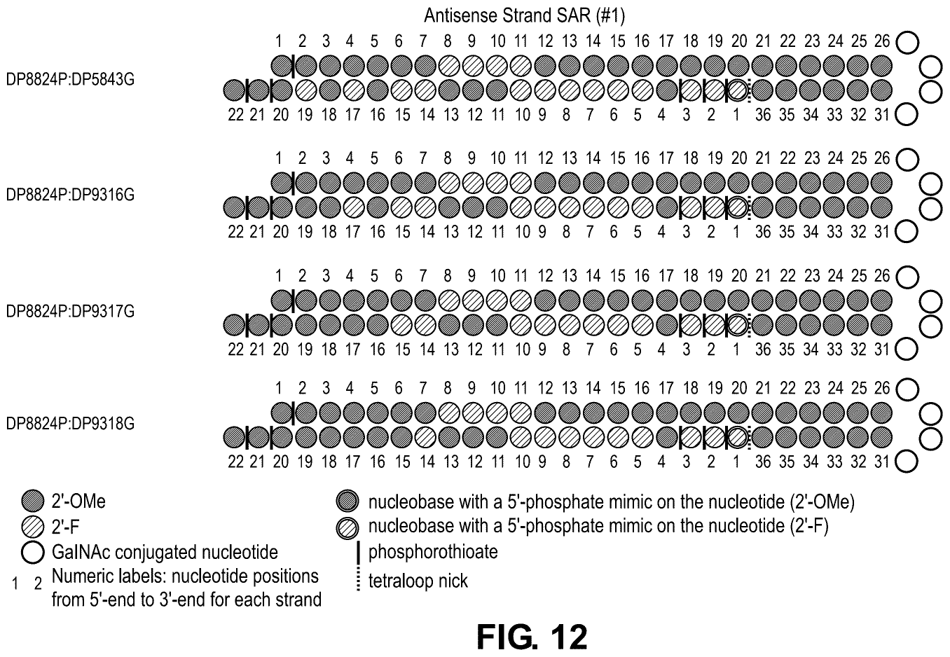

As shown in FIG. 12, the antisense strand was investigated by replacing 2′-F with 2′-OMe at positions 15, 17, and 19 on the antisense strand. Modifications of the sense strand of the dsRNA were kept constant in this analysis (FIG. 12). Similar potency was determined for each of the tested dsRNA as shown in FIGS. 2A-2D. Taken together, these results demonstrate that 2′-OMe modifications are well tolerated at positions 15, 17, and 19 of the antisense strand of the dsRNA.

### Example 3: Antisense Strand Analyzed by Replacing 2′-F with 2′-OMe at Positions 1-10

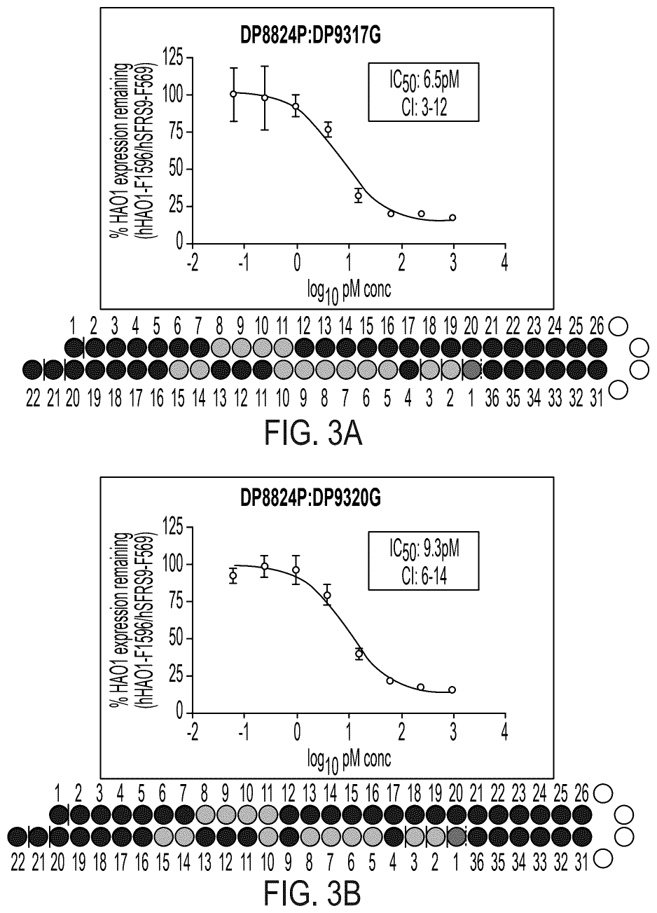

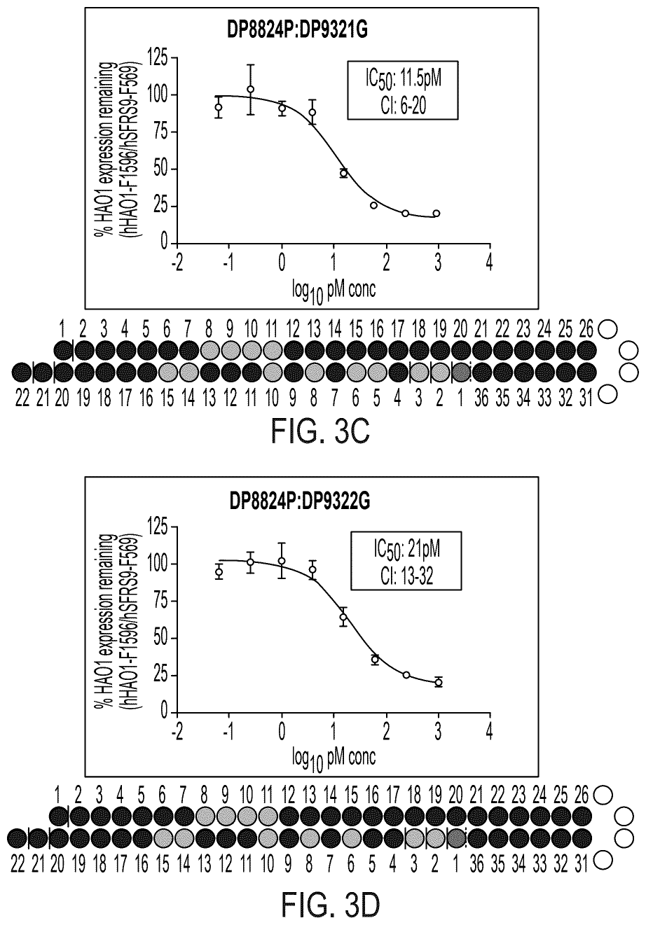

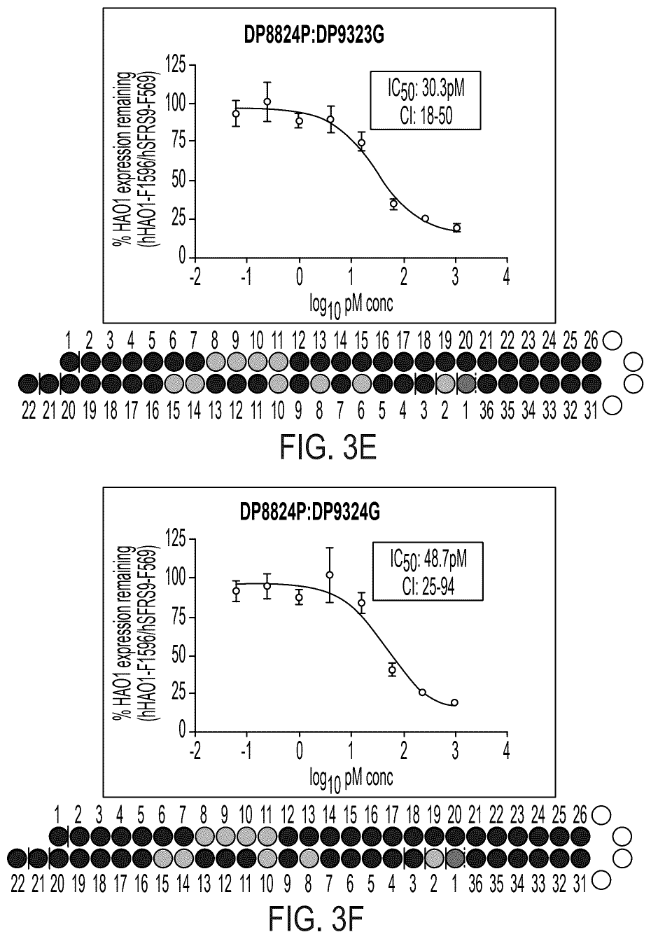

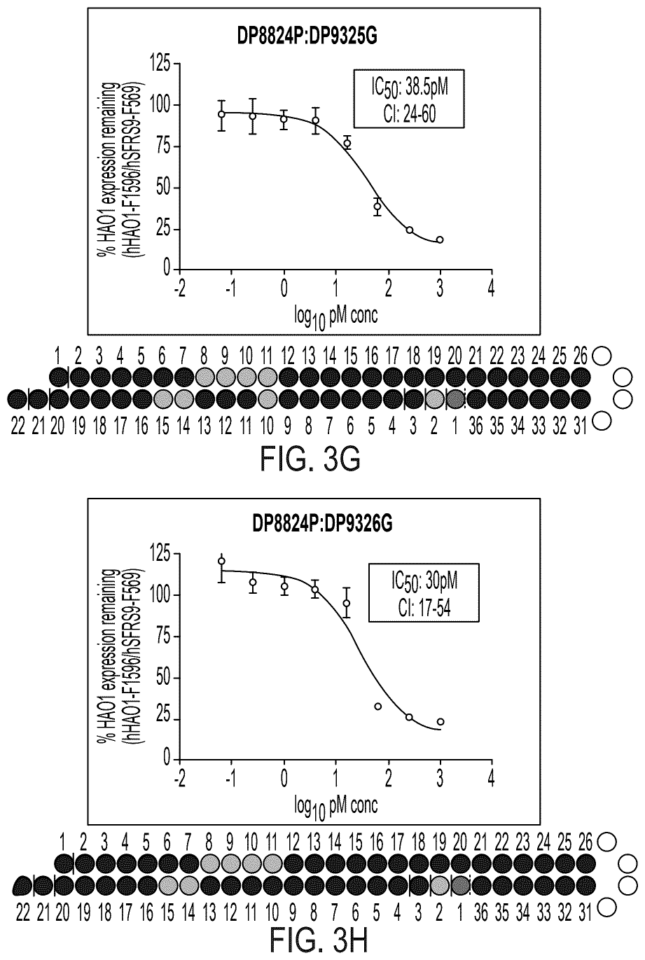

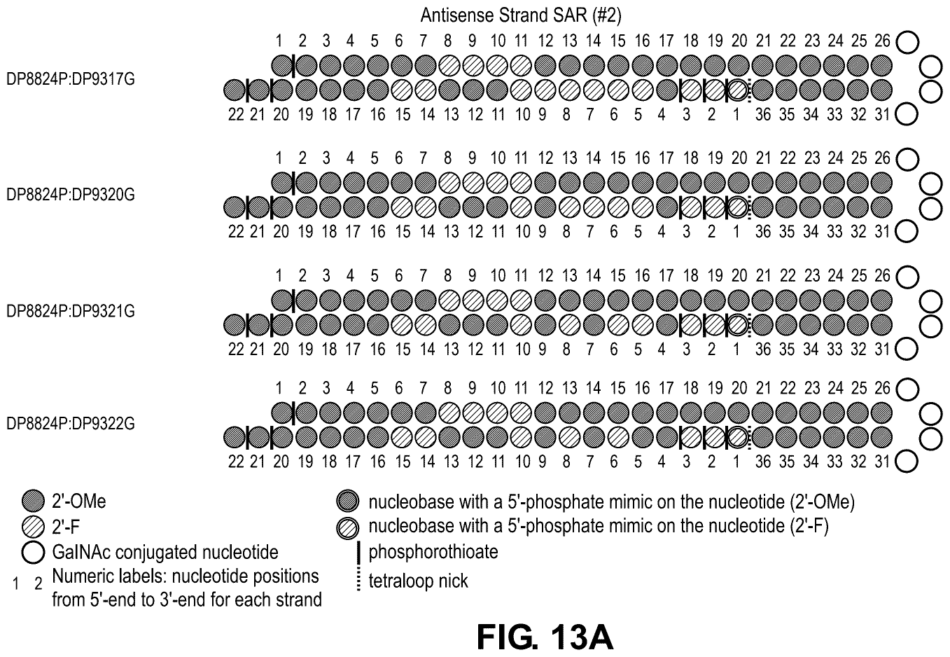

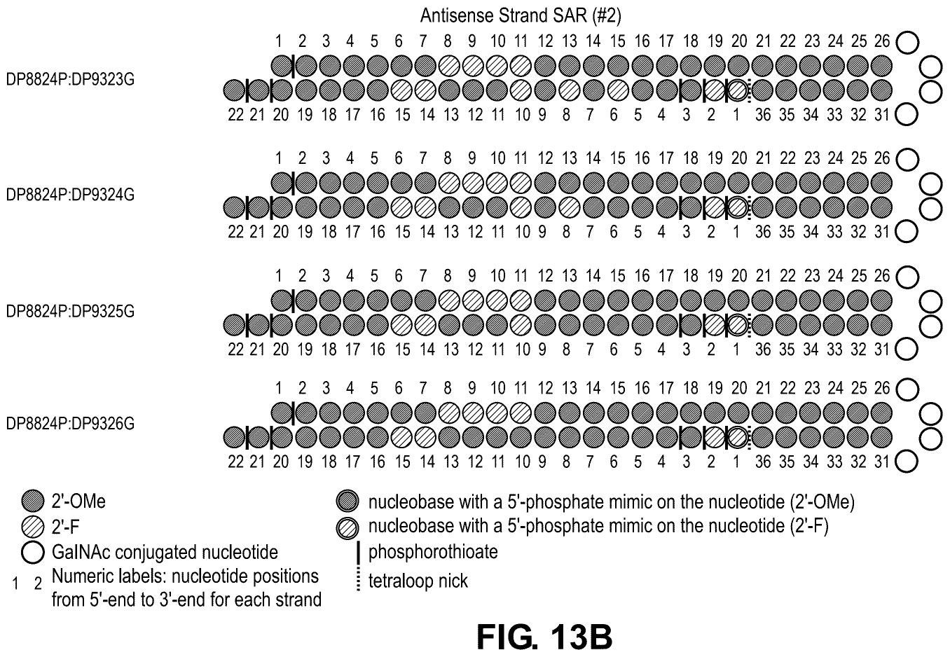

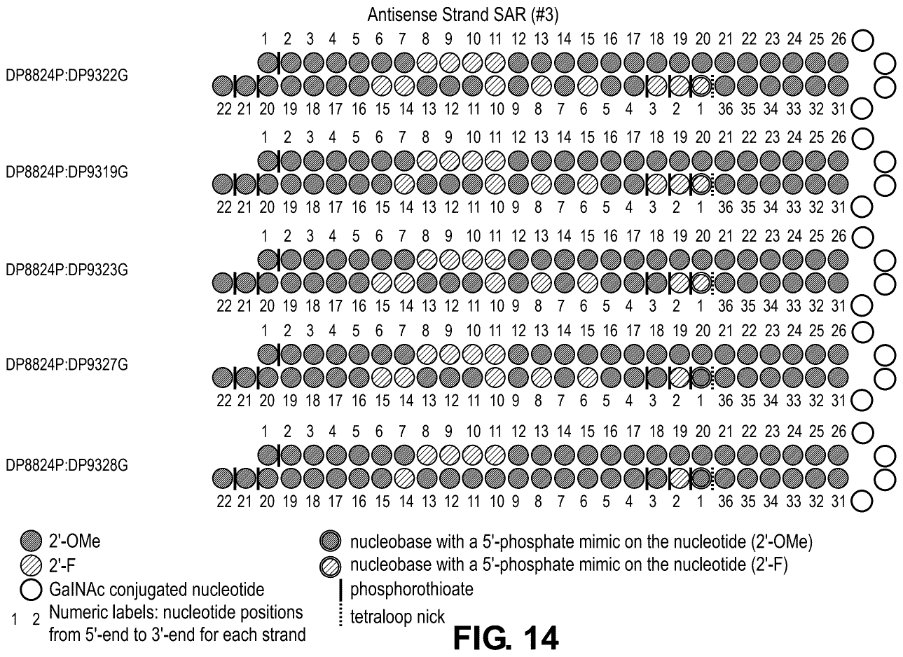

As shown in FIGS. 13A-13B, the antisense strand was investigated by replacing 2′-F with 2′-OMe at positions 1-10 on the antisense strand, also referred to as the seed region. As shown in FIGS. 3A-3H, 2′-OMe modifications at positions 7 and 9 were well tolerated. However, as 2′-F modifications are replaced with 2-OMe at positions 2 and 5, and at other positions in the seed region, the RNAi potency as determined by IC₅₀ value decreased (FIGS. 3A-3G). Taken together, the results demonstrate that 2′-OMe is poorly tolerated at the seed region of the antisense strand, and that position 5 prefers modification with 2′-F over 2′-OMe.

### Example 4: Antisense Strand Analyzed by Replacing 2′-F with 2′-OMe at Positions 1, 3, 6, 8, 10, and 15

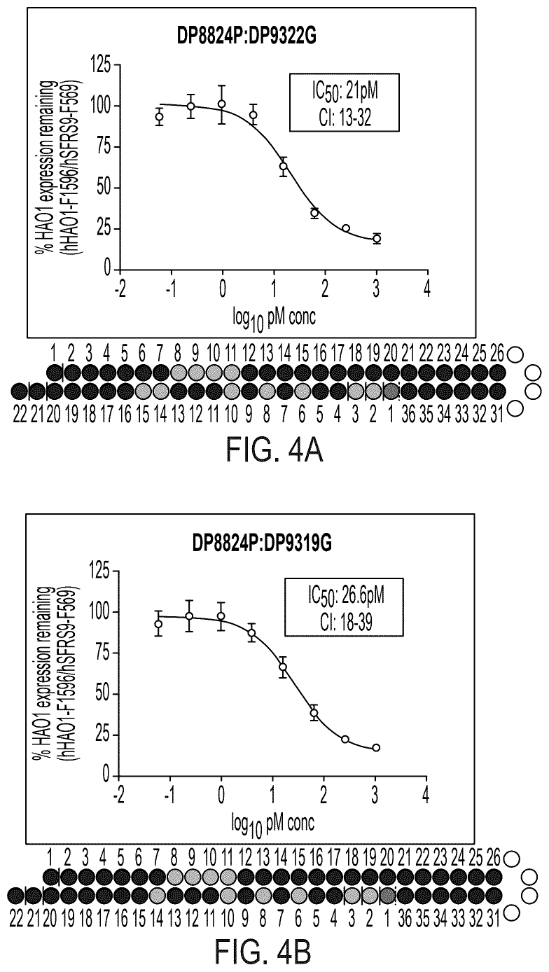

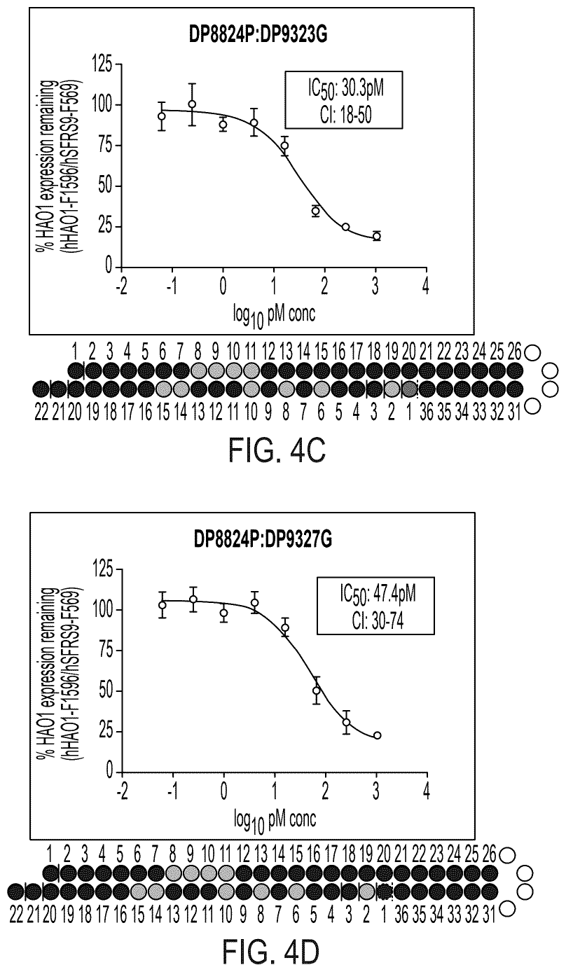

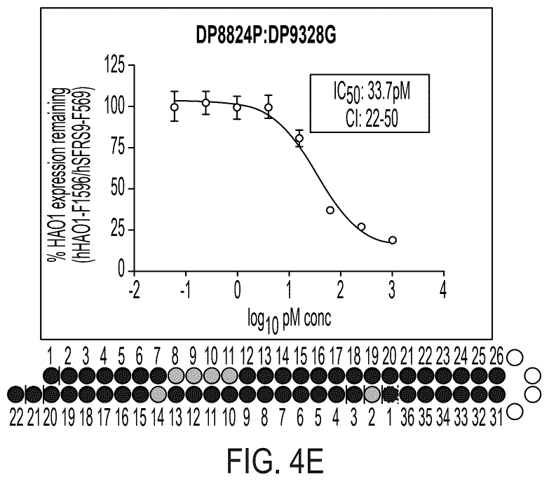

As shown in FIG. 14, the antisense strand was investigated by replacing 2′-F with 2′-OMe at positions 1, 3, 6, 8, 10, and 15 on the antisense strand. As shown in FIGS. 4A-4E, 2′-OMe modification at position 15 was well tolerated, which was consistent with results obtained in Example 2. The effect of 2′ modification on position 1 of the antisense strand, which contains a phosphate mimic on the 5′-end, was examined. Similar potency between 2′-OMe and 2′-F on position 1 were observed (FIGS. 4C-4D). Next, the effect 2′ modification on only positions 2 and 14 of the antisense strand was examined, and similar IC₅₀ values were obtained as compared to others tested (FIGS. 4A-4E). Taken together, the results demonstrate that 2′-OMe is tolerated on the antisense strand.

### Example 5: Antisense Strand Analyzed by Addition of 2′-F at Positions 3-6

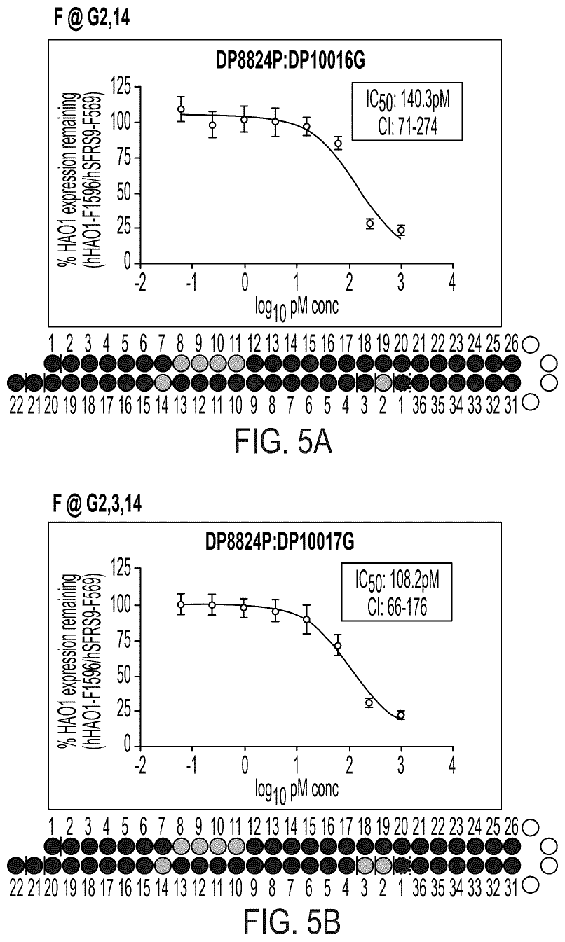

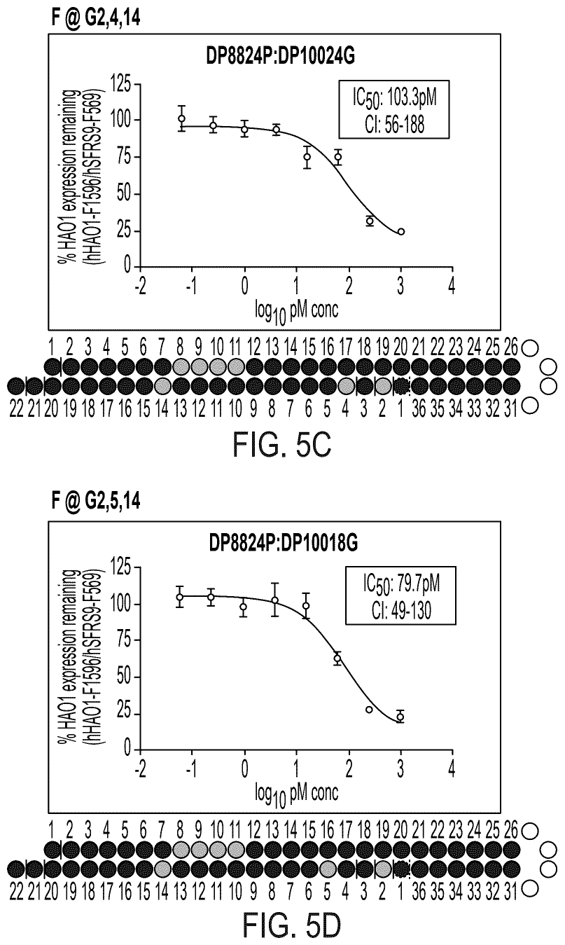

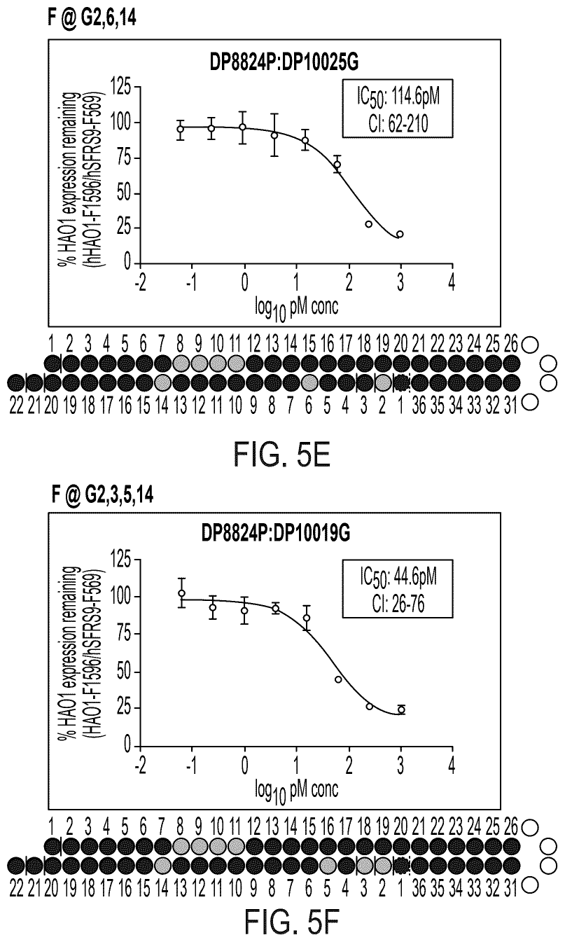

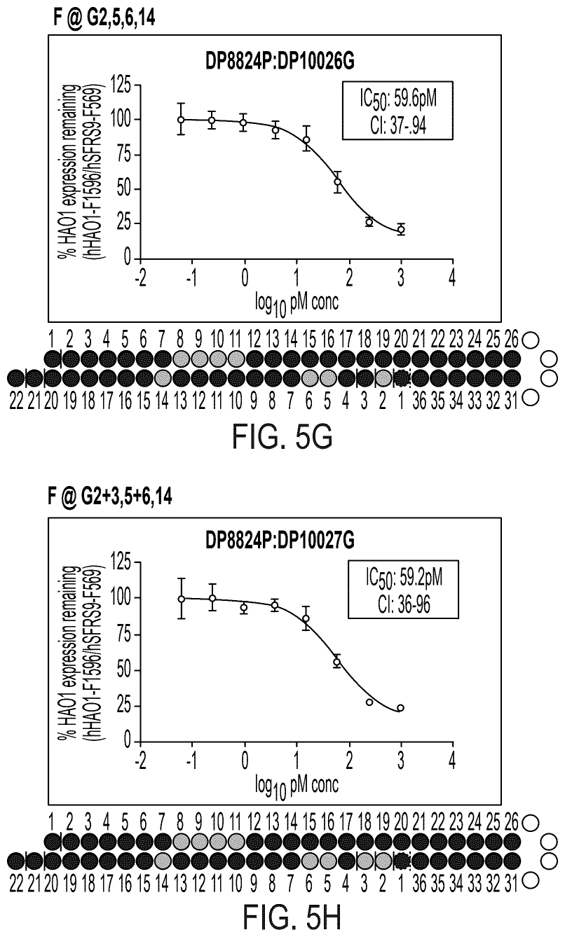

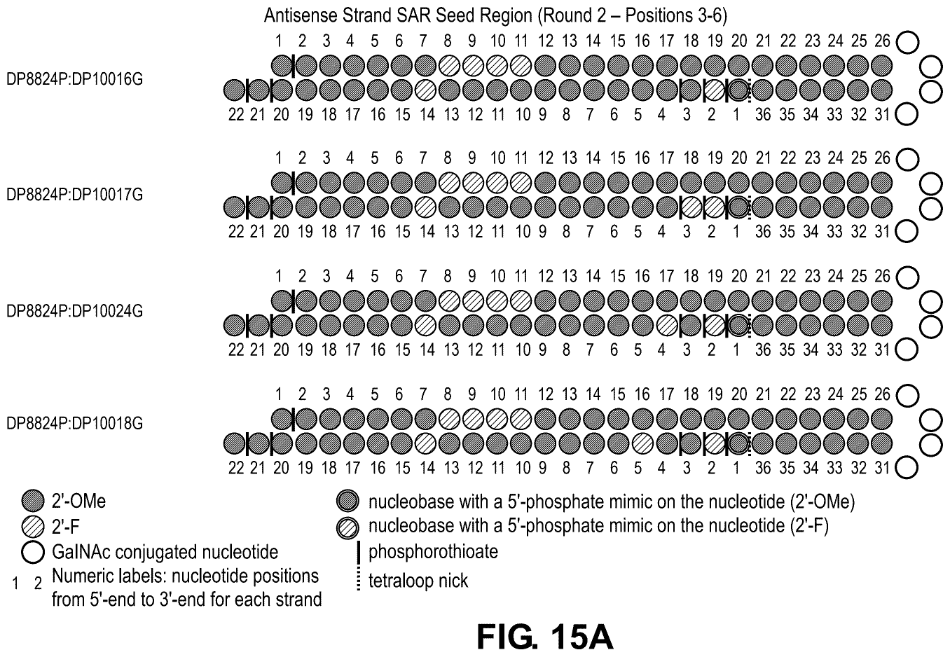

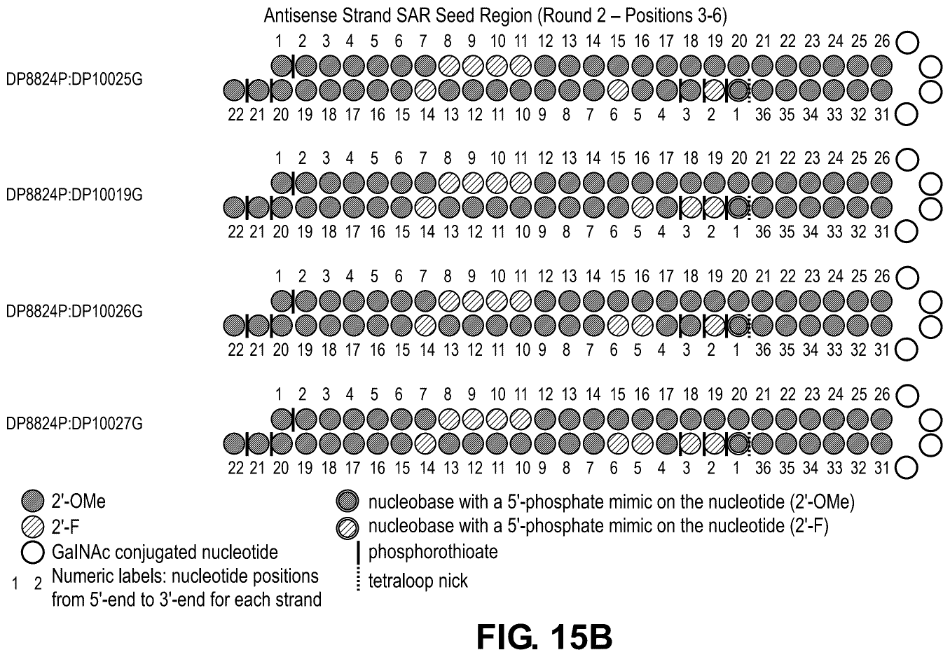

Next, a low 2′-F pattern (2′-F at positions 2 and 14 only of the antisense strand) was chosen as the starting point, and 2′-F was gradually added in the seed region at positions 3-6 to probe the sensitivity in that region. As shown in FIGS. 15A-15B, the starting molecule had the same modification pattern as the last molecule shown in FIG. 14 except that the molecules contain different phosphate mimics on antisense position 1. Based on the IC₅₀ results, 2′-F modification at position 5 showed an increase in potency compared to 2′-F modification at positions 3, 4, and 6 (FIGS. 5A-5H). These result further confirmed that position 5 may prefer 2′-F over 2′-OMe in some low 2′-F patterns. Furthermore, increased potency was observed when 2′-F on position 5 was tested in combination with 2′-F on other positions, such as 2′-F at position 3 or position 6 (FIGS. 5A-5H).

### Example 6: Antisense Strand Analyzed by Replacing 2′-F with 2′-OMe at Positions 7 to 10, and Maintaining 2′-F at Positions 3 and 5

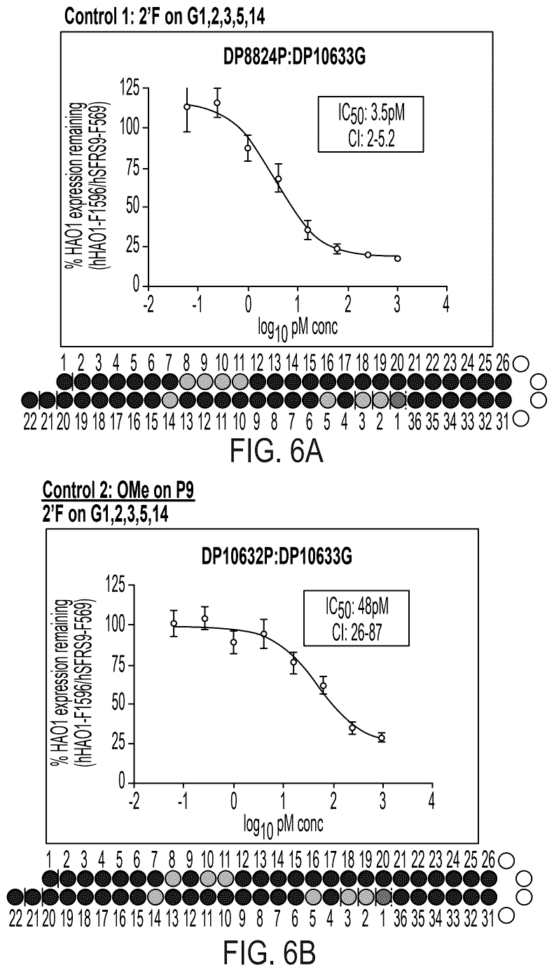

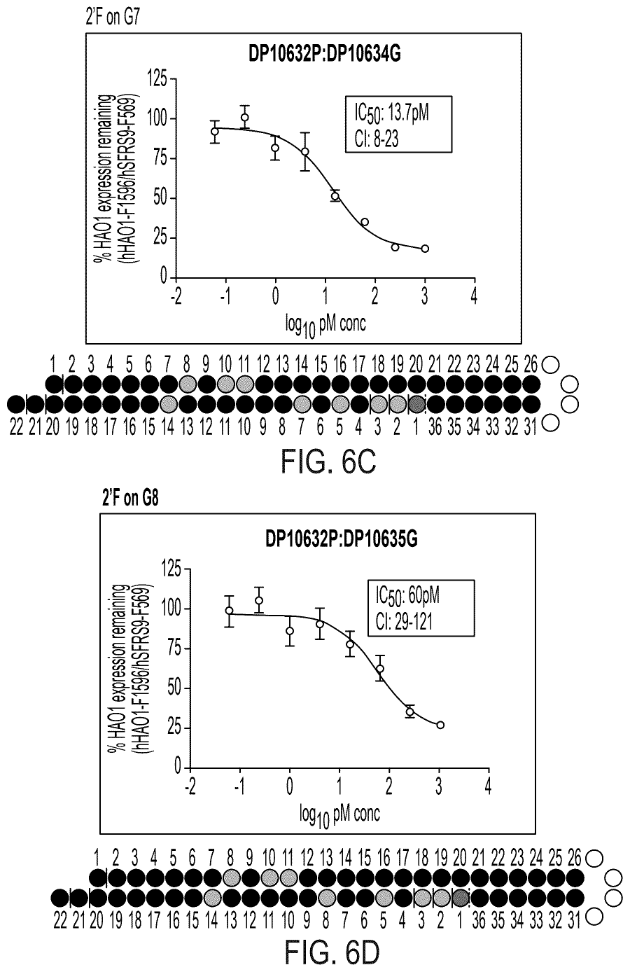

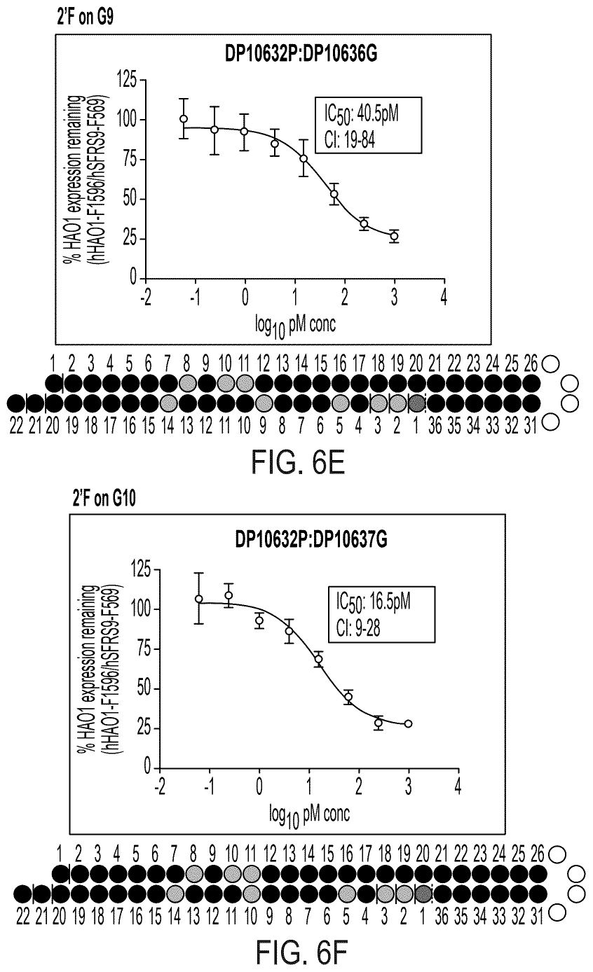

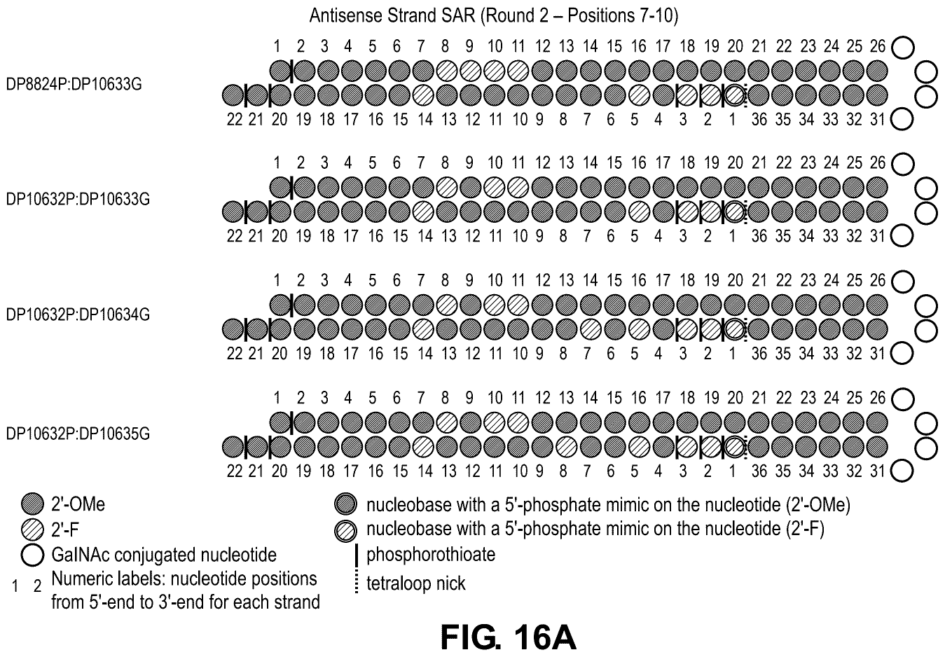

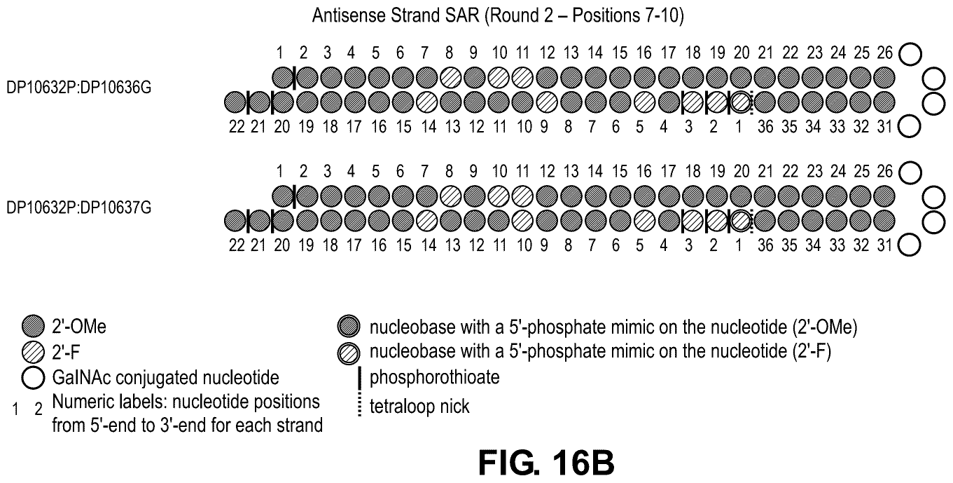

Next, positions 7 to 10 on the antisense strand were investigated (FIGS. 16A-16B). In this analysis, 2′-F modification was maintained at positions 5 and 3, and a phosphate mimic with 2′-F modification was maintained on position 1. As shown in FIG. 6A, control 1 showed an excellent IC₅₀ (3.5 pM) after 66 hrs of transfection in the HAO1 stable cell line. In order to probe the impact of 2′-F on positions 7 to 10, 2′-OMe was added on position 9 of the sense strand. This modification will provide a wider dynamic range for examination of the changes in IC₅₀s. As shown in FIG. 6, the IC₅₀ of control 2 is >10 fold higher than control 1 (FIGS. 6A-6B). As 2′-F was substituted on positions 7 through 10, an increase in potency was observed (FIGS. 6A-6F). The results showed that the potency was improved with 2′-F modification on position 7 or position 10, but not with 2′-F on position 8 or position 9.

### Example 7: Minimal 2′-F Set for HAO1 In Vivo Study

Taken together, the potency experimental results proved herein demonstrated that the antisense strand is more sensitive to 2′-OMe modifications than the sense strand. Positions on the antisense strand that preferred 2′-F over 2′-OMe were identified, which included positions 2, 3, 5, 7, 10, and 14. Among positions 3, 5, 7, and 10, position 5 was more pronounced in its preference for 2′-F over 2′-OMe. Modification patterns on the just noted positions may provide opportunities to balance potency, duration, and tolerability. The experimental results also showed that the sense strand can tolerate more 2′-OMe modifications than the antisense strand. Further, positions 8-11 on the sense strand preferred 2′-F over 2′-OMe, yet 2′-OMe insertion in this region was tolerated, especially when combined with optimal modifications on the antisense strand.

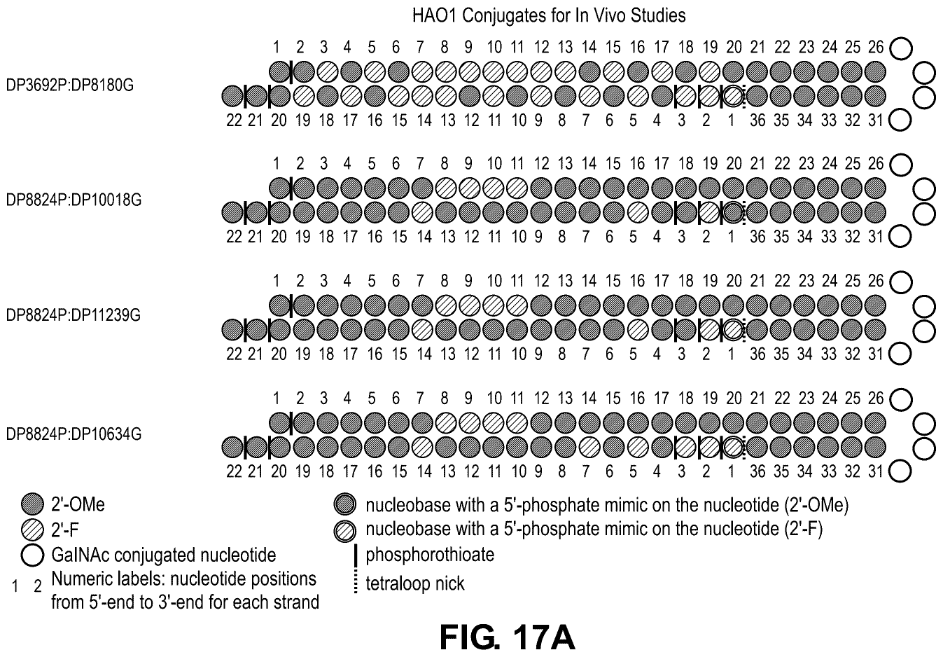

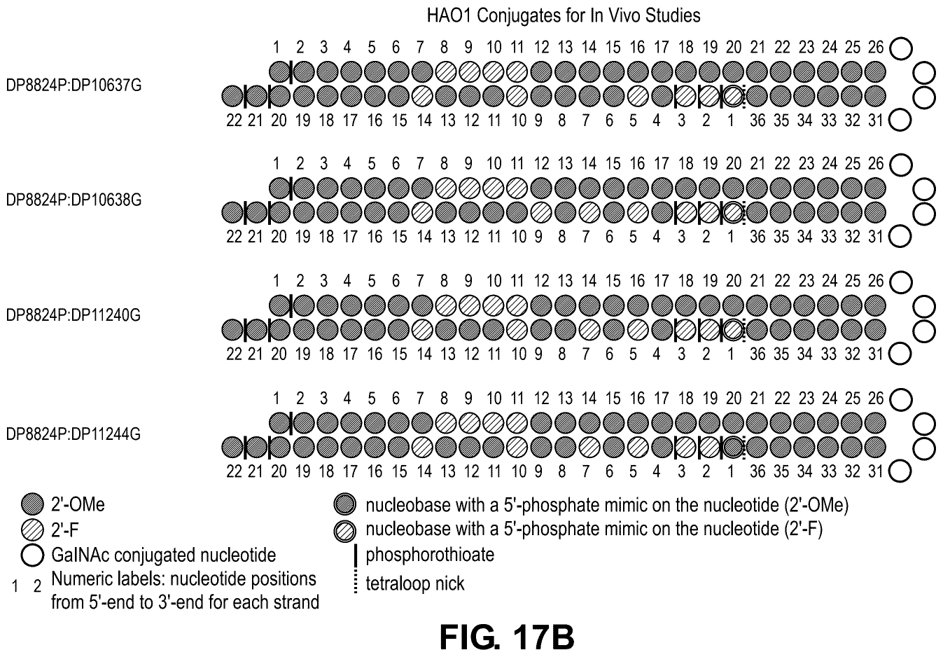

To test the in vivo activity of HAO1 conjugates comprising minimal 2′-F and heavy 2′-OMe modification patterns, mice were administered the HAO1 conjugates, and target knockdown was evaluated. HAO1 conjugates tested in mice are shown in FIGS. 17A-17B. A HAO1 conjugate comprising heavy 2′-F was used as a control.

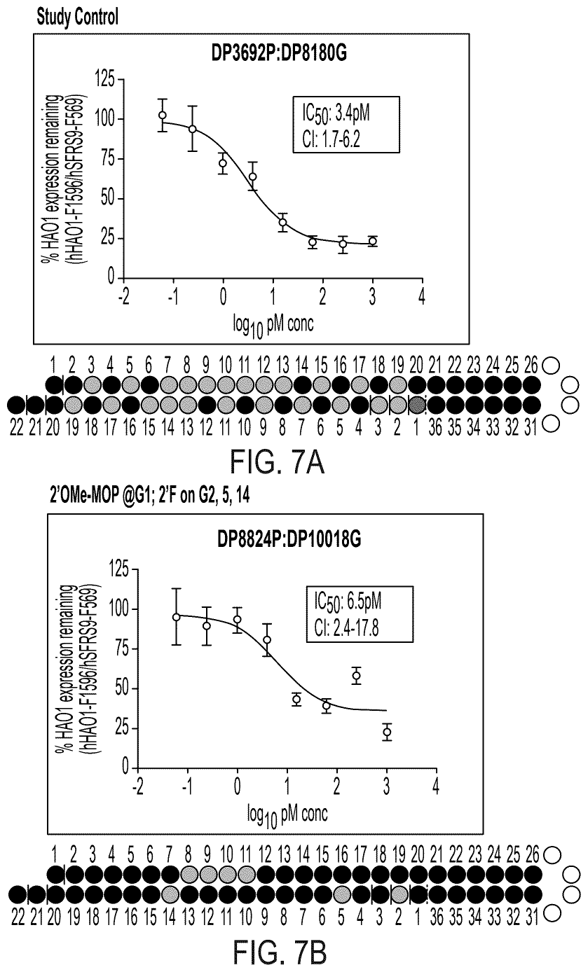

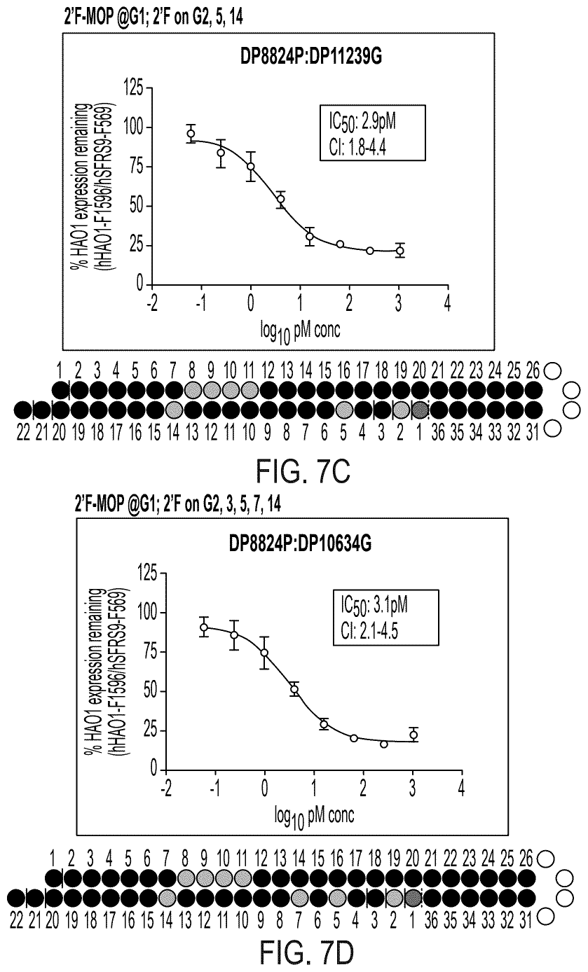

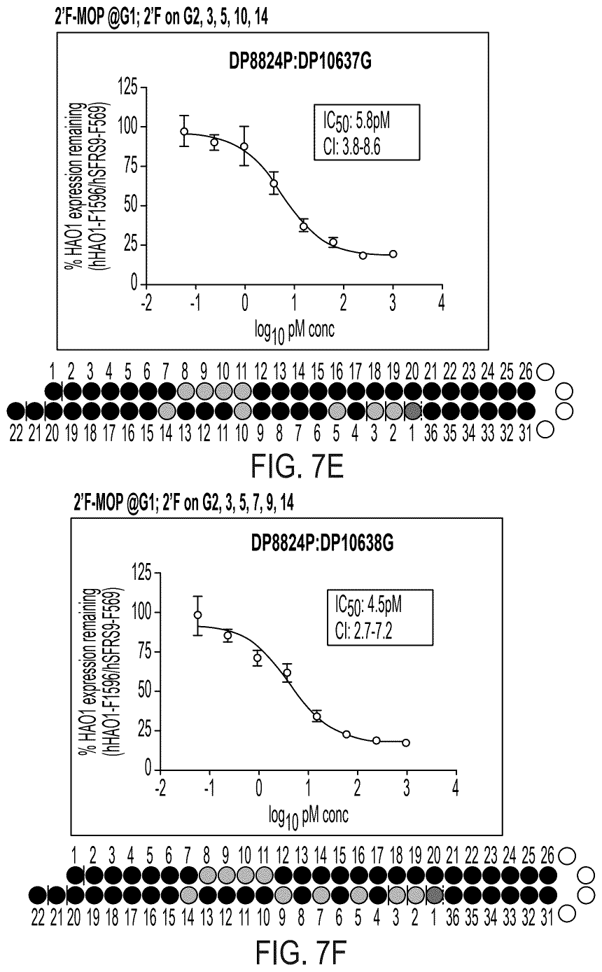

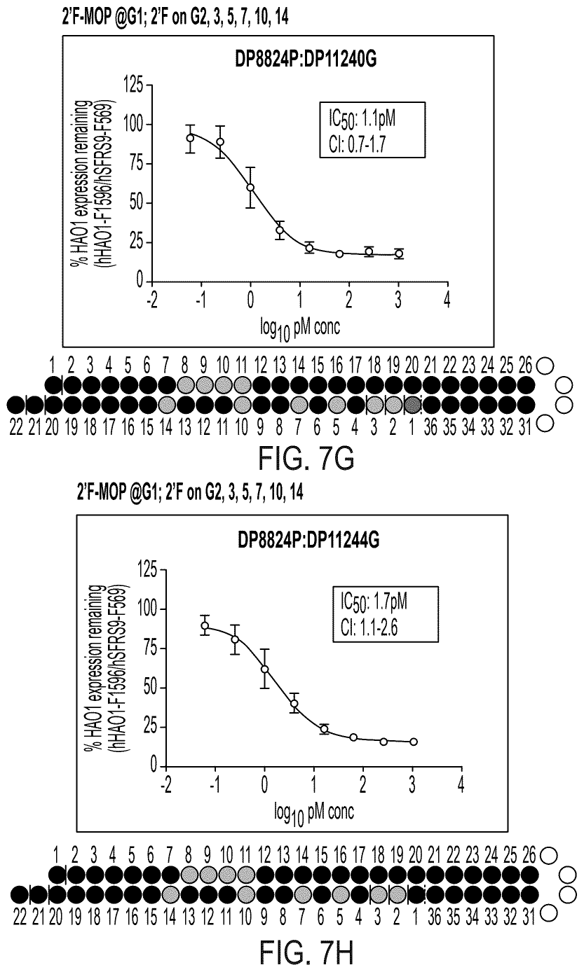

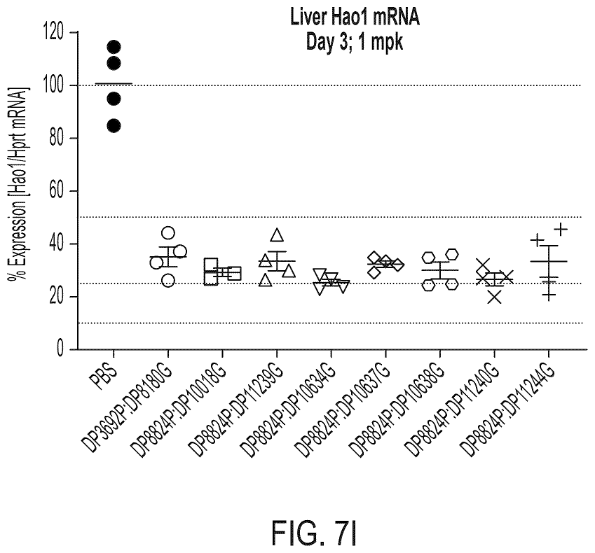

As shown in FIGS. 7A-7H, HAO1 conjugates comprising minimal 2′-F and heavy 2′-OMe modification patterns showed excellent potency (IC50s) in vitro in the HAO1 stable cell line, and their IC50s were comparable to the heavy 2′-F control. The HAO1 conjugates shown in FIGS. 17A-17B were also administered to mice by subcutaneous injection of a single dose of 1 mpk. Liver HAO1 mRNA expression relative to the PBS control group was measured 3 days post dose. As shown in FIG. 7I, the HAO1 conjugates comprising minimal 2′-F and heavy 2′-OMe modification patterns showed comparable KD activities in vivo compared to those of the heavy 2′-F control. No difference was detected between either 2′-F or 2′-OMe modifications in combination with a phosphate mimic on position 1 of the antisense strand. No difference was observed at day 3 for the comparison of 2′-OMe vs 2′-F on antisense position 1 in combination with a phosphate mimic. These results demonstrated a correlation between the in vitro and in vivo activities of the HAO1 conjugates comprising minimal 2′-F and heavy 2′-OMe modification patterns described herein.

### Example 8: HAO1 Duration Study

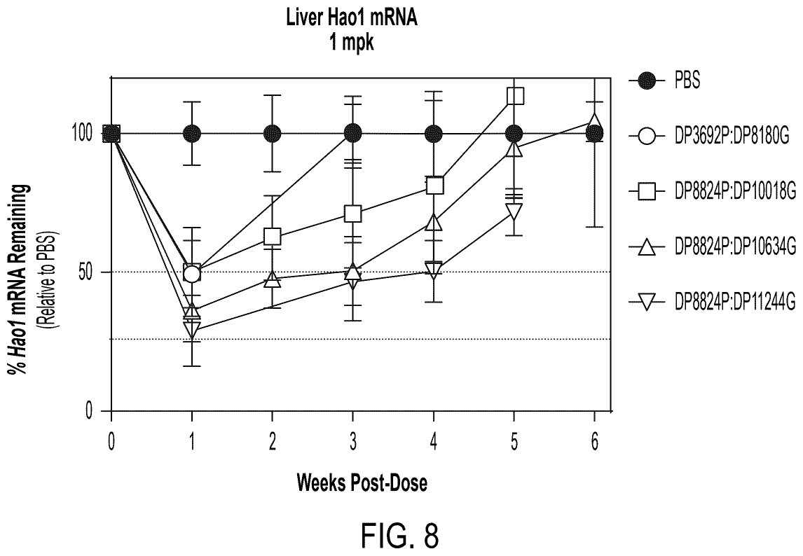

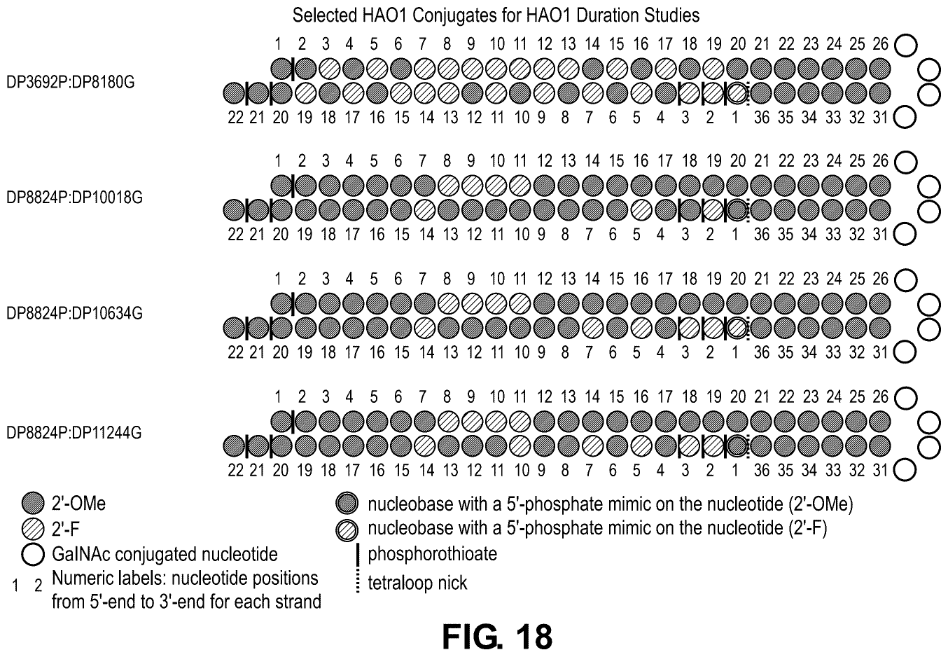

Modification with 2′-OMe typically provides better metabolic stability toward nuclease degradation than modification with 2′-F. Therefore, minimal 2′-F and heavy 2′-OMe modified nucleic acids should last longer in the cell. To test whether nucleic acids modified with 2′-OMe persist longer in the cell, duration studies were conducted using selected HAO1 conjugates test in the previous in vivo study (FIG. 18). As shown in FIG. 8, minimal 2′-F and heavy 2′-OMe modified nucleic acids showed better mRNA knockdown at longer time points, and therefore, better duration of RNAi activity in vivo, as compared to the heavy 2′-F control.

### Example 9: APOC3 Conjugates Having Minimal 2′-F and Heavy 2′-OMe Modifications

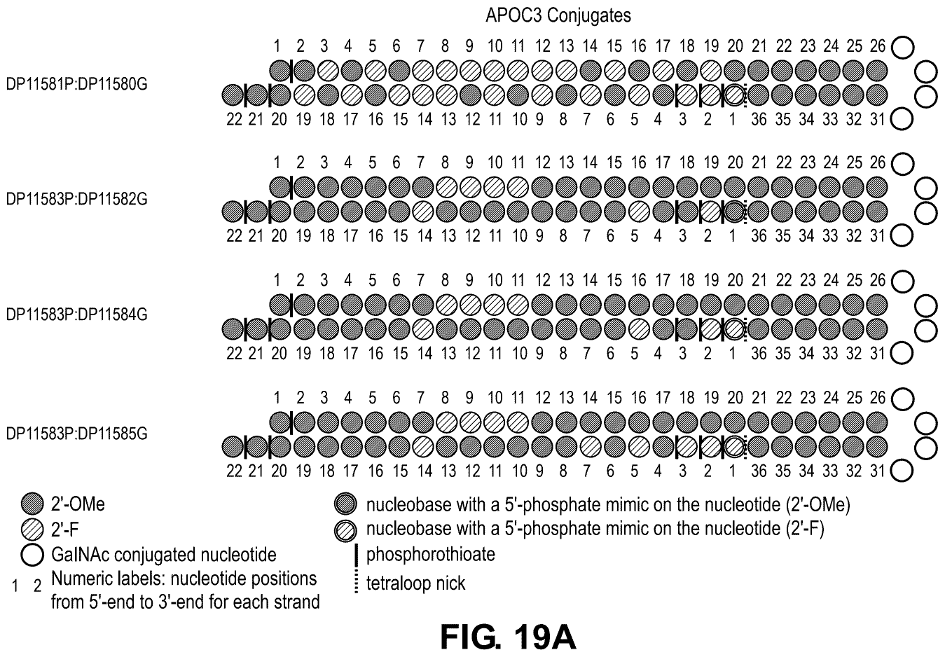

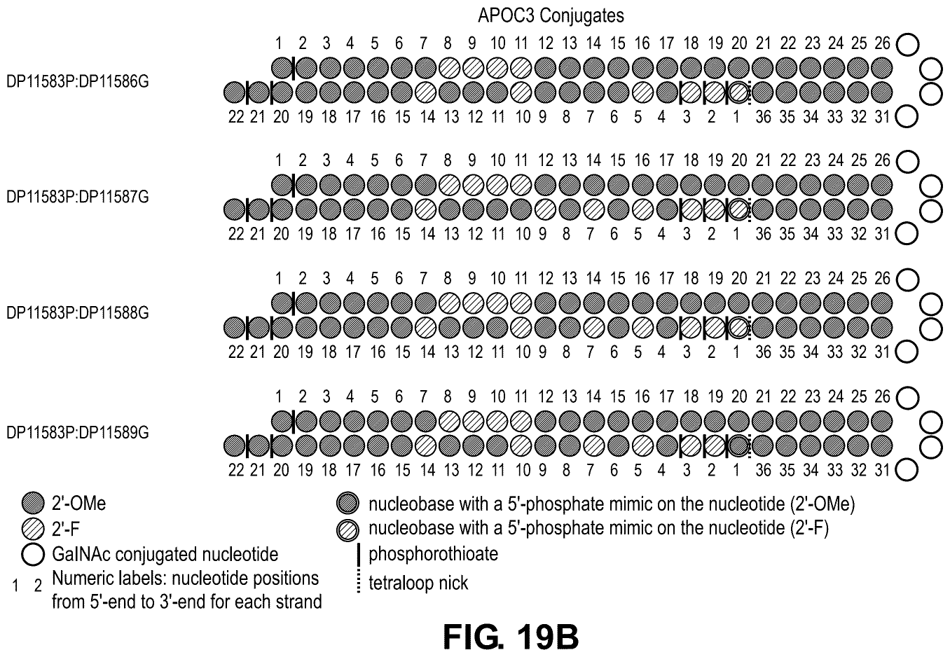

To confirm that nucleic acids having minimal 2′-F and heavy 2′-OMe modification patterns can be applied to other target sequences, modification patterns of the HAO1 conjugates shown in FIGS. 17A-17B were transferred onto an APOC3 sequence. The resulting APOC3 conjugates shown in FIGS. 19A-19B were tested in vitro and in vivo.

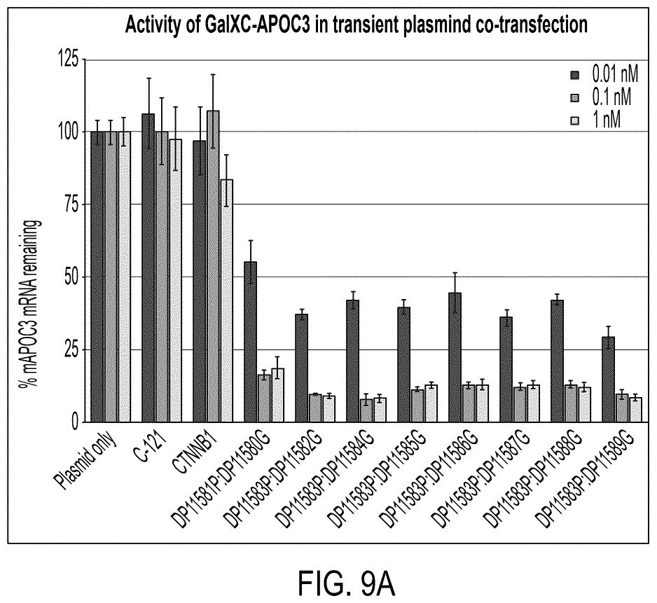

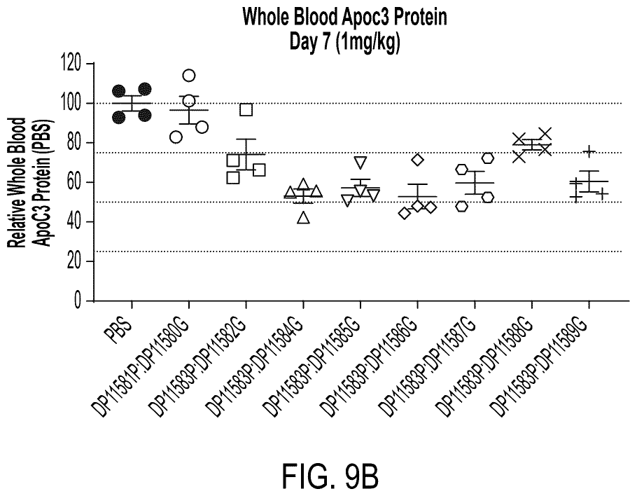

For in vitro experiments, HEK-293 cells were co-transfected with 100 ng of pcDNA3-mAPOC3 plasmid (containing cDNA for mouse APOC3) and siRNAs at the indicated concentration using Dharmafect Duo reagent (Dharmacon) according to the manufacturer's protocol. The next day the cells were lysed and RNA was purified using the SV96 kit (Promega). The purified RNA was reverse transcribed using High-capacity RT kit (Life Technologies) and APOC3 cDNA was quantified at RT-qPCR using gene assays for mouse APOC3, normalized against human SFRS9. As shown in FIG. 9, APOC3 conjugates having minimal 2′-F and heavy 2′-OMe modification patterns were well tolerated and showed similar in vitro activity as compared to the heavy 2′-F control.

For in vivo experiments, CD-1 mice were divided into study groups and were dosed subcutaneously with 1 mg/kg of the assigned APOC3 conjugate. Animals were bled on day 7 post dose via lateral tail vein puncture with a collection volume of 10 μL. Collected whole blood was diluted immediately 1:5000 in cold PBS, and subsequently frozen at −20° C. Whole blood at a final dilution of 1:10,000 was used for determining plasma APOC3 levels using the Cloud Clone Corporation ELISA (SEB890Mu). As seen in FIG. 9, APOC3 conjugates having minimal 2′-F and heavy 2′-OMe modification patterns showed good activity while the heavy 2′-F control did not show activity on day 7 post dose.

### Example 10: GYS2 Conjugates Having Minimal 2′-F and Heavy 2′-OMe Modifications

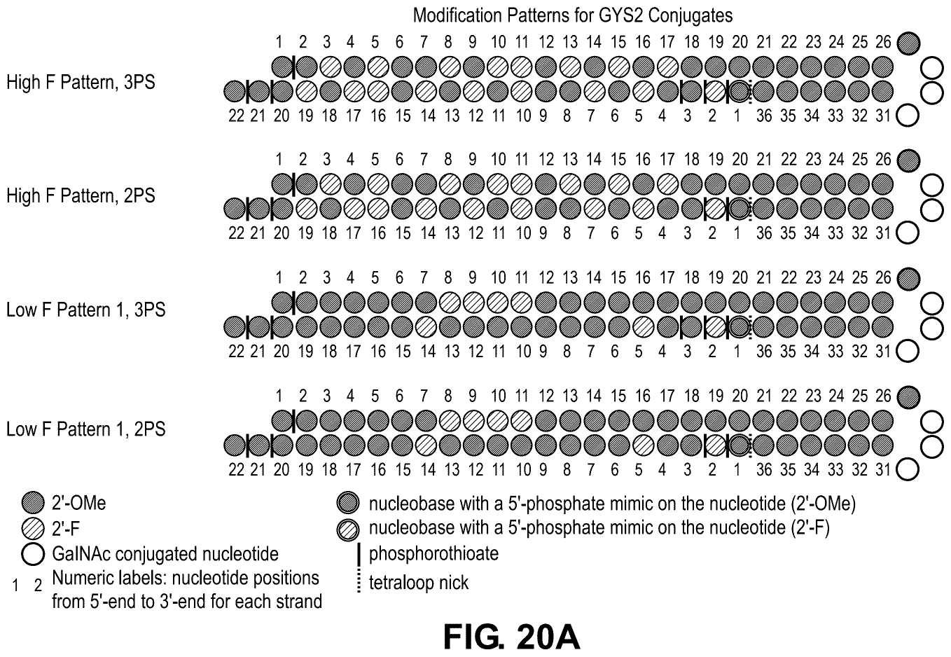

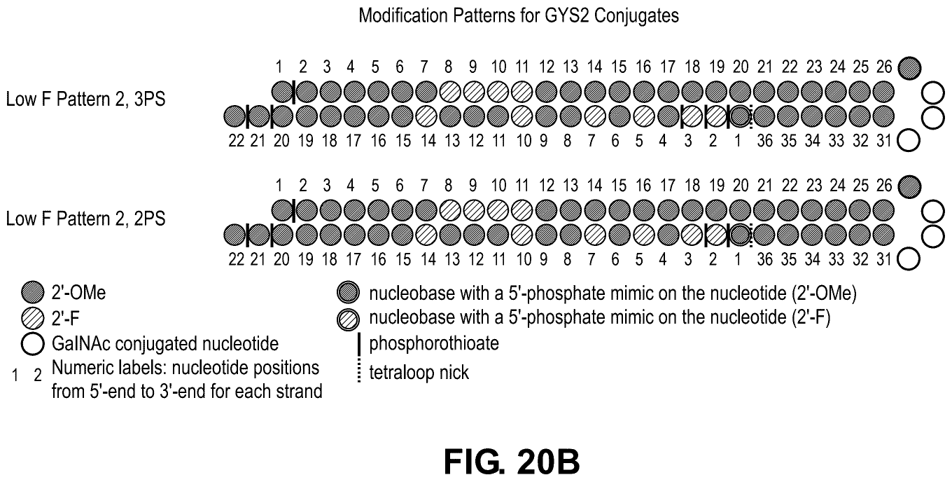

To confirm that nucleic acids having minimal 2′-F and heavy 2′-OMe modification patterns can be applied to other target sequences, modification patterns of the HAO1 conjugates shown in FIGS. 17A-17B were transferred onto different GYS2 sequences. The resulting GYS2 conjugates are shown in FIGS. 20A-20B. Two minimal 2′-F patterns were chosen and compared to a heavy 2′-F pattern (FIGS. 20A-20B). For each of the three patterns, either 3 phosphorothioates (3PS) or 2 phosphorothioates (2PS) were included on the 5′-end of the antisense strand. GYS2 conjugates contained 3 GalNAc conjugated nucleotides in the loop region. Four different GYS2 sequences comprising the patterns in FIGS. 20A-20B were tested.

As shown in FIG. 10, minimal 2′-F and heavy 2′-OMe modification patterns 1 and 2 were well tolerated in vivo compared to the heavy 2′-F control, specifically these patterns were tolerated 4 days after a single subcutaneous dose of 0.5 mg/kg. Similar results were obtained for each of the four GYS2 sequences tested.

In sum, several advanced tetraloop GalXC designs were developed with reduced 2′-F content and increased 2′-OMe content that can be applied to multiple target genes and sequences with optimal potency and duration.

1-30. (canceled)

31. An oligonucleotide comprising:
a sense strand comprising 17-36 nucleotides, wherein the sense strand has a first region (R1) and a second region (R2), wherein the second region (R2) of the sense strand comprises a first subregion (S1), a second subregion (S2) and a tetraloop (L) or triloop (triL) that joins the first and second regions, wherein the first and second subregions form a second duplex (D2);
an antisense strand comprising 20-22 nucleotides, wherein the antisense strand includes at least 1 single-stranded nucleotide at its 3′-terminus, wherein the sugar moiety of the nucleotide at position 5 of the antisense strand is modified with a 2′-F and the sugar moiety of each of the remaining nucleotides of the antisense strand is modified with a modification selected from 2′-O-propargyl, 2′-O-propylamin, 2′-amino, 2′-ethyl, 2′-aminoethyl (EA), 2′-fluoro (2′-F), 2′-O-methyl (2′-OMe), 2′-O-methoxyethyl (2′-MOE), 2′-O-[2-(methylamino)-2-oxoethyl] (2′-O-NMA), or 2′-deoxy-2′-fluoro-β-d-arabinonucleic acid (2′-FANA), and wherein the sense strand and antisense strand are separate strands; and
a first duplex (D1) formed by the first region of the sense strand and the antisense strand, wherein the first duplex has a length of 12-20 base pairs and has 7-10 nucleotides that are modified at the 2′-position of the sugar moiety with 2′-F.

32. The oligonucleotide of claim 31, wherein the sugar moiety at positions 2 and 14 of the antisense strand is modified with 2′-F.

33. The oligonucleotide of claim 32, wherein the sugar moiety at each of up to 3 nucleotides at positions 1, 3, 7, and 10 of the antisense strand is additionally modified with 2′-F.

34. The oligonucleotide of claim 31, wherein the sugar moiety of each of the nucleotides at positions 8-11 of the sense strand is additionally modified with 2′-F.

35. The oligonucleotide of claim 31, wherein the sugar moiety of each of the nucleotides at positions 1-7 and 12-17 or 12-20 of the sense strand are modified with a modification selected from 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), or 2′-deoxy-2′-fluoro-β-d-arabinonucleic acid (2′-FANA).

36. The oligonucleotide of claim 31, wherein:
the sugar moiety of each of the nucleotides at positions 2, 5, and 14 of the antisense strand is modified with 2′-F; or
the sugar moiety of each of the nucleotides at positions 1, 2, 5, and 14 of the antisense strand is modified with 2′-F, or
the sugar moiety of each of the nucleotides at positions 1, 2, 3, 5, 7, and 14 of the antisense strand is modified with 2′-F; or
the sugar moiety of each of the nucleotides at positions 2, 3, 5, 7, and 14 of the antisense strand is modified with 2′-F; or
the sugar moiety of each of the nucleotides at positions 1, 2, 3, 5, 10, and 14 of the antisense strand is modified with 2′-F; or
the sugar moiety of each of the nucleotides at positions 2, 3, 5, 10, and 14 of the antisense strand is modified with 2′-F; or
the sugar moiety of each of the nucleotides at positions 2, 3, 5, 7, 10, and 14 of the antisense strand is modified with 2′-F; and
the sugar moiety of each of the remaining nucleotides of the antisense strand is modified with a modification selected from 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), or 2′-deoxy-2′-fluoro-β-d-arabinonucleic acid (2′-FANA).

37. The oligonucleotide of claim 31, wherein the antisense strand has 3 nucleotides that are modified at the 2′-position of the sugar moiety with 2′-F.

38. The oligonucleotide of claim 31, wherein the second duplex has a length of 1-6 base pairs.

39. The oligonucleotide of claim 31, wherein the second duplex comprises at least one bicyclic nucleotide.

40. The oligonucleotide of claim 39, wherein the second duplex has a length of 1-3 base pairs.

41. The oligonucleotide of claim 31, wherein:
the triloop has a nucleotide sequence of GAA or AAA; or
the tetraloop is an RNA tetraloop selected from GAAA, UNCG, GNRA, or CUUG or a DNA tetraloop selected from d(GNAB), d(CNNG), or d(TNCG), wherein N is any one of U, A, C, G and R is G or A.

42. The oligonucleotide of claim 31, wherein the sugar moiety of each nucleotide in the second duplex is modified with 2′-O-methyl (2′-OMe).

43. The oligonucleotide of claim 31, wherein 1-3 nucleotides in the triloop or 1-4 nucleotides in the tetraloop are conjugated to a ligand.

44. The oligonucleotide of claim 43, wherein the ligand comprises N-acetylgalactosamine.

45. The oligonucleotide of claim 31, wherein the nucleotide at position 1 of the antisense strand comprises a phosphate mimic.

46. The oligonucleotide of claim 31, wherein the sense strand comprises 36 nucleotides and the antisense strand comprises 22 nucleotides.

47. A single-stranded oligonucleotide comprising 20-22 nucleotides, wherein:
the sugar moiety of each of the nucleotides at positions 2, 5, and 14 and optionally up to 3 of the nucleotides at positions 1, 3, 7, and 10 of the antisense strand is modified with 2′-F; and
the sugar moiety of each of the remaining nucleotides of the antisense strand is modified with a modification selected from 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), or 2′-deoxy-2′-fluoro-β-d-arabinonucleic acid (2′-FANA).

48. A pharmaceutical composition comprising the oligonucleotide of claim 31 and a pharmaceutically acceptable carrier.

49. A method for reducing expression of a target gene in a subject, or treating a disease or disorder in a subject, comprising administering the oligonucleotide of claim 31 to the subject in an amount sufficient to reduce expression of a target gene in the subject.

50. An oligonucleotide comprising:
a sense strand comprising 17-36 nucleotides, wherein the sense strand has a first region (R1) and a second region (R2), wherein the second region (R2) of the sense strand comprises a first subregion (S1), a second subregion (S2) and a tetraloop (L) or triloop (triL) that joins the first and second regions, wherein the first and second subregions form a second duplex (D2);
an antisense strand comprising 20-22 nucleotides, wherein the antisense strand includes at least 1 single-stranded nucleotide at its 3′-terminus, wherein the sugar moiety of the nucleotide at position 5 of the antisense strand is modified with a 2′-F and the sugar moiety of each of the remaining nucleotides of the antisense strand is modified with a modification selected 2′-O-propargyl, 2′-O-propylamin, 2′-amino, 2′-ethyl, 2′-aminoethyl (EA), 2′-fluoro (2′-F), 2′-O-methyl (2′-OMe), 2′-O-methoxyethyl (2′-MOE), 2′-O-[2-(methylamino)-2-oxoethyl] (2′-O-NMA), or 2′-deoxy-2′-fluoro-β-d-arabinonucleic acid (2′-FANA), and wherein the sense strand and antisense strand are separate strands; and
a first duplex (D1) formed by the first region of the sense strand and the antisense strand, wherein the first duplex has a length of 12-20 base pairs and has 10 nucleotides that are modified at the 2′-position of the sugar moiety with 2′-F.
